(19) United States
(12) Patent Application Publication
(10) Pub. No.: US 2023/0375549 A1
(43) Pub. Date: Nov. 23, 2023

(54) PD-1 AS A PREDICTIVE MARKER FOR THERAPY IN CANCER

(71) Applicant: PRELUDE CORPORATION, Laguna Hills, CA (US)

(72) Inventors: Axel Emanuel Stenmark Tullberg, Göteborg (SE); Erik Carl Viktor Holmberg, Asperö (SE); Per Oswald Karlsson, Göteborg (SE); Troy M. Bremer, Irvine, CA (US)

(21) Appl. No.: 17/998,334

(22) PCT Filed: May 12, 2021
(86) PCT No.: PCT/US21/32080

§ 371 Date: Aug. 11, 2023

Related U.S. Application Data
(60) Provisional application No. 63/169,532, filed on Apr. 1, 2021.

(30) Foreign Application Priority Data
May 13, 2020 (SE) 2050559-0

(51) Int. Cl. G01N 33/574

(52) U.S. Cl. CPC G01N 33/57415; G01N 2333/70532; G01N 2800/52

(57) ABSTRACT

Provided are embodiments for treating breast cancer comprising obtaining a tissue sample of a tumour from a breast cancer patient, determining an expression level of PD-1 in the sample, determining that the expression level is below a threshold level, providing intensified treatment to the subject. The intensified treatment can be intensified radiotherapy treatment.

### INCORPORATION BY REFERENCE TO ANY PRIORITY APPLICATIONS

Any and all applications for which a foreign or domestic priority claim is identified in the Application Data Sheet as filed with the present application are hereby incorporated by reference under 37 CFR 1.57.

### REFERENCE TO SEQUENCE LISTING, TABLE, OR COMPUTER PROGRAM LISTING

The present application is being filed along with a Sequence Listing in electronic format. The Sequence Listing is provided as a file entitled SequenceListingPRLUD.010WO.txt, created May 11, 2021 which is 8,192 bytes in size. The information in the electronic format of the Sequence Listing is incorporated herein by reference in its entirety.

### BACKGROUND

### Field

The present technology generally relates to whether or not a subject who has breast cancer will be responsive to standard radiotherapy in terms of local recurrence of breast cancer.

### Description of the Related Art

There are a variety of markers for the identification of tumors in subjects. In addition, there are various markers that can be used for the prediction of neoplastic progression. For example, U.S. Pat. Pub. Nos. 2010/0003189, 2012/0003639, and 20170350895 disclose a variety of markers that when examined in various combinations can predict the likelihood that a subject will have DCIS and/or invasive breast cancer.

The protein PD-1 is as so called “immune checkpoint” of the immune system. Its discovery as a drug target for the treatment of cancer was rewarded with the Nobel prize in medicine in 2018. PD-1 inhibitory antibodies, such as Keytruda, (Pembrolizumab) is a recent treatment for many cancer forms. PD-1 expression in a cancer tumour generally correlates with poor prognosis (Shen, T., et al., Sci Rep, 2017. 7(1): p. 7848, Jin, S., et al Oncotarget, 2017. 8(24): p. 38850-38862, Jiang, F., et al. BMC Cancer, 2019. 19(1): p. 503). PD-1 overexpression is characteristic of a dysfunctional antitumoral immune response. In breast cancer, increased levels of PD-1 expressing immune cells are associated with a worse overall survival (Muenst, S., et al., Breast Cancer Res Treat, 2013. 139(3): p. 667-76). PD-1 expression is also positively correlated with unfavourable clinicopathological characteristics such as larger tumour size, higher histological grade (more aggressive tumour characteristics) and cancer cells in the lymph nodes (Muenst, S., et al., Breast Cancer Res Treat, 2013. 139(3): p. 667-76). However, none of these items address an appropriate therapy for the subject being examined.

### SUMMARY

As disclosed herein, it has been surprisingly found that low PD-1 expression indicates that the patient belongs to a subgroup where intensified treatment, such as for example, intensified radiotherapy treatment is useful to the patient.

In a first aspect, provided herein is a method for treating breast cancer (both invasive and in situ) comprising the steps:
a) obtaining a tissue sample of a tumour from a breast cancer patient,
b) determining the expression level of PD-1 in the sample,
c) determining that the expression level is below a threshold level,
d) providing intensified treatment as radiotherapy treatment, systemic therapy or mastectomy to the patient.

This provides a new and efficient way of determining the level of radiotherapy that is beneficial to the patient. It is desirable to avoid exposing the patient to more radiation than necessary and high doses of radiation should only be provided to patients for which it is necessary. The results are particularly surprising because generally high PD-1 expression correlates with poor prognosis.

In some embodiments the intensified treatment comprises intensified radiotherapy treatment, preferably whole breast external radiotherapy, accelerated partial breast radiotherapy or brachytherapy or a combination thereof, with a biologically effective dose of (BED) of 80 Gy or more. In some embodiments, the calculation of BED is based on the formula BED=D(1+d/(α/β)) where D is the total dose in Gy, d is the dose per fraction in Gy and 60 /β is the characteristic constant of the tissue being referred to. Typically, α/β=4 for breast cancer. A person skilled in the field of breast radiotherapy is familiar with the concept of BED and how BED is determined for different treatment protocols.

In some embodiments the intensified treatment comprises systemic therapy. The patient may have been subjected to breast conserving surgery or total mastectomy.

In some embodiments, the breast cancer may be early stage breast cancer.

In some embodiments, the level of PD-1 expression may be determined in any suitable manner, for example by detecting the amount of PD-1 mRNA in the sample. The mRNA sequence may comprise the nucleotide sequence of SEQ ID NO 1.

In a second aspect of the invention there is provided a PD-1 mRNA-binding nucleotide for use in the diagnosis of breast cancer, where the nucleotide is used for quantifying the level of PD-1 that is expressed in a breast cancer sample, and where low expression of PD-1 indicates that the patients belong to a patient subgroup where intensified radiotherapy treatment is needed.

In a third aspect of the invention there is provided a method of diagnosis comprising the steps of:
a) obtaining a tissue sample of a tumour from a breast cancer patient,
b) determining the expression level of PD-1 in the sample,
c) determining that the expression level is below a threshold expression level, determining that the patient belongs to a group that would benefit from intensified radiotherapy treatment.

In some embodiments, a method of treating a subject is provided. The method comprises identifying an incremental risk to a subject with invasive breast cancer or in situ breast cancer of a local or regional recurrence of an invasive breast cancer based on a level of PD-1 in a sample of an invasive breast cancer in the subject; and administering an intensified breast cancer therapy to the subject based upon the incremental risk, wherein a higher incremental risk will increase:
a) a likelihood of an aggressive breast cancer therapy that is at least more than what would be recommended by the NCCN;
b) the aggressiveness of the aggressive breast cancer; or
c) both a) and b).

In some embodiments, a method for treating a subject for recurrence of invasive breast cancer is provided. The method comprises: providing a cancer tissue sample from a subject who has invasive breast cancer; analyzing the cancer tissue sample for a level of PD-1; and treating the subject with an intensified treatment if the cancer tissue sample has a low level of PD-1.

In some embodiments, a method of treating a subject is provided. The method comprises: identifying a subject with invasive breast cancer that has a low level of PD-1; and administering an intensified treatment to the invasive breast cancer.

In some embodiments, a method for recommending a treatment to a subject is provided. The method comprises analyzing a cancer tissue sample for a level of PD-1 from a subject; and recommending that one treat the subject with an intensified treatment if the cancer tissue sample has a low level of PD-1.

In some embodiments, a method for preventing an invasive breast cancer recurrence in a subject is provided. The method comprises: providing a cancer tissue sample from a subject who has invasive breast cancer; analyzing the cancer tissue sample for a level of PD-1; and administering an intensified treatment if the cancer tissue sample has a low level of PD-1.

In some embodiments, a method for preventing an invasive breast cancer recurrence in a subject is provided. The method comprises receiving an intensified treatment if a cancer has a low level of PD-1.

In some embodiments, a method of modifying a treatment for a subject is provided. The method comprises identifying a subject with invasive breast cancer that has a low level of PD-1; and administering a breast cancer therapy to the subject, wherein the breast cancer therapy is more aggressive than a traditional breast cancer therapy, wherein the traditional breast cancer therapy is one recommended for the subject, based on the subject's risk factors excluding PD-1 levels.

In some embodiments, a method of selecting a treatment for a subject is provided. The method comprises: comparing a level of PD-1 in a subject to a range of PD-1 levels; and increasing a likelihood of administering radiotherapy to the subject as an inverse function of a level of PD-1, wherein a lower PD-1 level indicates a greater benefit of radiotherapy to the subject, thereby decreasing a risk of local breast cancer recurrence.

In some embodiments, any of the methods herein can be applied for assistance in determining the effectiveness of radiotherapy for local cancer recurrence.

### BRIEF DESCRIPTION OF THE DRAWINGS

FIG. 1 shows two graphs that show ipsilateral breast tumor recurrence (IBTR) for patients with high and low PD1 expression, respectively. Solid line=no radiotherapy, dotted line =radiotherapy. As used herein, this references mRNA levels.

FIG. 2 shows two graphs that show breast cancer (BC) recurrence for patients with high and low PD1 expression, respectively. Solid line=no radiotherapy, dotted line=radiotherapy. As used herein, this references mRNA levels.

FIGS. 3A-3E shows graphs demonstrating the various results of the cut-offs across various percentiles of the population.

FIG. 4 presents the various sequences identified as SEQ ID NOs 1-4 provided herein.

FIG. 5 are boxplots that show the expression of housekeeping genes and PD1 (aka PDCD1) relative to one another. Such housekeeping gene levels can be used as a comparator to PD-1 levels so that relevant ratios between the various genes and PD-1 can be compared to across different subjects.

FIG. 6A-6L are histograms of various reference genes.

FIG. 6A are histograms of YWHAZ, TRAP1, and UBC.

FIG. 6B are histograms of YWHAG, SRSF4, and TBP.

FIG. 6C are histograms of TFRC, PUM1, and RPL4.

FIG. 6D are histograms of RPLP2, SDHA, and POLR2A.

FIG. 6E are histograms of PPIA, PPIB, and MAPRE2.

FIG. 6F are histograms of PEX16, PGK1, and GUSB.

FIG. 6G are histograms of HMBS, HPRT1, and HSP90AB1.

FIG. 6H are histograms of FPGS, GAPDH, and GINS2.

FIG. 6I are histograms of DECR1, DIMT1, and EEF1A1.

FIG. 6J are histograms of FARP1, CCK, and CRY2.

FIG. 6K are histograms of CSNK1G2, A4GALT, and ACTB.

FIG. 6L is histogram of B2M.

FIG. 7 is histogram of PDCD1 (aka PD-1).

### DETAILED DESCRIPTION OF VARIOUS EMBODIMENTS

Provided herein are embodiments relating to methods and compositions for examining PD-1 levels. By determining PD-1 levels within a tumor of a subject, one can determine if standard radiotherapy (RT) will work in the subject who has invasive breast cancer. An appropriate therapy can then be implemented in a variety of ways. In some embodiments, one can avoid (or instruct accordingly) to not administer standard RT at all (no radiotherapy). Instead, the subject can receive an alternative to standard radiotherapy. In some embodiments, one can administer a non-standard level of radiotherapy (e.g., an especially high level of radiotherapy). Thus, in some embodiments, subjects with low PD-1 levels will not receive standard radiotherapy, and can instead receive either a) an alternative to standard radiotherapy and/or b) especially high doses of radiotherapy (e.g. intense or aggressive therapy) because standard radiotherapy will not likely work for the subject. In some embodiments, the effectiveness of the therapy is in the context of a local recurrence of the cancer.

### Definitions and Optional Embodiments

The term “and/or” shall be taken to provide explicit support for both meanings or for either meaning.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

The following explanations of terms and methods are provided to better describe the present disclosure and to guide those of ordinary skill in the art in the practice of the present disclosure. The singular forms “a,” “an,” and “the” refer to one or more than one, unless the context clearly dictates otherwise. For example, the term “comprising a nucleic acid molecule” includes single or plural nucleic acid molecules and is considered equivalent to the phrase “comprising at least one nucleic acid molecule.” The term “or” refers to a single element of stated alternative elements or a combination of two or more elements, unless the context clearly indicates otherwise. As used herein, “comprises” means “includes.” Thus, “comprising A or B,” means “including A, B, or A and B,” without excluding additional elements. Unless otherwise specified, the definitions provided herein control when the present definitions may be different from other possible definitions.

Unless explained otherwise, all technical and scientific terms used herein have the same meaning as commonly understood to one of ordinary skill in the art to which this disclosure belongs. All HUGO Gene Nomenclature Committee (HGNC) identifiers (IDs) mentioned herein are incorporated by reference in their entirety. Although methods and materials similar or equivalent to those described herein can be used in the practice or testing of the present disclosure, suitable methods and materials are described below. The materials, methods, and examples are illustrative only and not intended to be limiting.

The term “array” denotes an arrangement of molecules, such as biological macromolecules (such as peptides or nucleic acid molecules) or biological samples (such as tissue sections), in addressable locations on or in a substrate. A “microarray” is an array that is miniaturized so as to require or be aided by microscopic examination for evaluation or analysis. Arrays are sometimes called chips or biochips.

The array of molecules makes it possible to carry out a very large number of analyses on a sample at one time. In some embodiments, arrays of one or more molecule (such as an oligonucleotide probe) will occur on the array a plurality of times (such as twice), for instance to provide internal controls. The number of addressable locations on the array can vary, for example from at least one, to at least 2, to at least 5, to at least 10, at least 20, at least 30, at least 50, at least 75, at least 100, at least 150, at least 200, at least 300, at least 500, least 550, at least 600, at least 800, at least 1000, at least 10,000, or more. In particular examples, an array includes nucleic acid molecules, such as oligonucleotide sequences that are at least 15 nucleotides in length, such as about 15-40 nucleotides in length. In particular examples, an array includes oligonucleotide probes or primers which can be used to detect the markers noted herein, such as PD-1.

In some embodiments, within an array, each arrayed sample can be addressable, in that its location can be reliably and consistently determined within at least two dimensions of the array. Addressable arrays can be computer readable, in that a computer can be programmed to correlate a particular address on the array with information about the sample at that position (such as hybridization or binding data, including for instance signal intensity). In some examples of computer readable formats, the individual features in the array are arranged regularly, for instance in a Cartesian grid pattern, which can be correlated to address information by a computer.

As used herein, the term “gene” means nucleic acid in the genome of a subject capable of being expressed to produce a mRNA in addition to intervening intronic sequences and in addition to regulatory regions that control the expression of the gene, e.g., a promoter or fragment thereof.

As used herein, the term “diagnosis”, and variants thereof, such as, but not limited to “diagnose” or “diagnosing” shall include, but not be limited to, a primary diagnosis of a clinical state or any primary diagnosis of a clinical state. A diagnostic assay described herein is also useful for assessing the remission of a subject, or monitoring disease recurrence, or tumor recurrence, such as following surgery, radiation therapy, adjuvant therapy or chemotherapy, or determining the appearance of metastases of a primary tumor.

In some embodiments, a prognostic assay described herein is useful for assessing likelihood of treatment benefit, disease recurrence, tumor recurrence, or metastasis of a primary tumor, such as following surgery, radiation therapy, adjuvant therapy or chemotherapy. All such uses of the assays described herein are encompassed by the present disclosure. In some embodiments, the test can be used to predict if the patient will have an occurrence.

The term “breast tumor” denotes a neoplastic condition of breast tissue that can be benign or malignant. The term “tumor” is synonymous with “neoplasm” and “lesion”. Exemplary breast tumors include invasive breast cancer, ductal carcinoma in situ (DCIS), lobular carcinoma in situ (LCIS), and atypical ductal hyperplasia (ADH).

The term “cancer” denotes a malignant neoplasm that has undergone characteristic anaplasia with loss of differentiation, increased rate of growth, invasion of surrounding tissue, and is capable of metastasis. The term “cancer” shall be taken to include a disease that is characterized by uncontrolled growth of cells within a subject, such as, but not limited to, invasive breast cancer. In some embodiments, invasion of the surrounding tissue is the invasion of the basement membrane.

The term “intraductal lesion” refers to tumors that are confined to the interior of the mammary ducts and are, therefore, not invasive breast cancers. Exemplary intraductal lesions include ADH and DCIS.

ADH is a neoplastic intraductal (non-invasive) lesion characterized by proliferation of evenly distributed, monomorphic mammary epithelial cells.

DCIS is a neoplastic intraductal (non-invasive) lesion characterized by increased mammary epithelial proliferation with subtle to marked cellular atypia. DCIS has been divided into grades (low, intermediate, and high) based on factors such as nuclear atypia, intraluminal necrosis, mitotic activity etc. Low-grade DCIS and ADH are morphologically identical, and ADH is distinguished from DCIS based on the extent of the lesion, as determined by its size and/or the number of involved ducts. DCIS is initially typically diagnosed from a tissue biopsy triggered by a suspicious finding (e.g., microcalcifications, unusual mass, tissue distortion or asymmetry, etc.) on a mammogram and/or ultrasound imaging test. It may be from routine screening imaging or, more rarely, from diagnostic imaging triggered by a positive physical examination (e.g., a palpable mass, nipple discharge, skin change, etc.) or by a significant change in a previously identified mass.

Cellular proliferation in DCIS is confined to the milk ducts. If the proliferating cells have invaded through the basement membrane of the myoepithelial cell (MEC) layer lining the duct, thus appearing in the surrounding stroma, then the lesion is considered an invasive breast cancer, even if DCIS is also present. In some cases, the invasion is very minimal (microinvasion) or the only evidence of invasion is disruption of the MEC layer (e.g., by observing discontinuities in MEC-specific protein marker stains such as SMMHC and/or p63). Typically, these microinvasive cases are treated as invasive breast cancers, although there is some controversy in the treatment of these cases.

Recurrence rates in DCIS with current treatments are difficult to estimate. However, it is likely that about 20% of patients who receive lumpectomies without any further treatment would experience recurrence events within 10 years, approximately evenly split between DCIS and invasive events, while <2% of patients who receive mastectomies would experience recurrence. Standard of care with lumpectomy is to receive adjuvant radiation therapy (RT). Several randomized clinical trials provide evidence that adjuvant radiation therapy following lumpectomy reduces recurrence risk by approximately half for both DCIS and invasive event types, and that current clinical and pathologic assessment techniques cannot identify a low-risk sub-group in which there is no benefit from radiation therapy.

LCIS is non-invasive lesion that originates in mammary terminal duct-lobular units generally composed of small and often loosely cohesive cells. When it has spread into the ducts, it can be differentiated from DCIS based on morphology and/or marker stains.

As used herein, “invasive breast cancer” denotes that the neoplastic (tumor) cells have invaded through the epithelial basement membrane. This distinguishes invasive breast cancer from other hyperplastic (ductal hyperplasia) or dysplastic (atypical ductal hyperplasia, ADH) or non-invasive neoplastic (DCIS, LCIS) breast lesions which are characterized by an intact (non-invaded) basement membrane. It can be divided into stages (I, IIA, IIB, IIIA, IIIB, and IV). In some embodiments, any of the methods provided herein can be applied to invasive breast cancer to determine the success of radiotherapy for preventing an invasive breast cancer recurrence. In some embodiments, any of the methods provided herein can be applied to DCIS to determine the success of radiotherapy for preventing a DCIS cancer recurrence.

Surgery is a treatment for a breast tumor and is frequently involved in diagnosis. The type of surgery depends upon how widespread the tumor is when diagnosed (the tumor stage), as well as the type and grade of tumor.

The term “treatment” as provided herein does not require the complete or 100% curing of the subject. Instead, it encompasses the broader concept or delaying the onset of one or more symptoms, extending the life and/or quality of life of the subject, reducing the severity of one or more symptoms, etc.

“Risk of invasive breast cancer”, denotes a risk of developing (or being diagnosed with) a subsequent invasive breast cancer in the same (a.k.a. ipsilateral) breast.

Adjuvant chemotherapy is often used after surgery to treat any residual disease. Systemic chemotherapy often includes a platinum derivative with a taxane. Adjuvant chemotherapy is also used to treat subjects who have a recurrence or metastasis.

“Adjuvant invasive breast cancer treatment” denotes any treatment that is appropriate for a subject that is likely to have an invasive breast cancer occurrence, which can include, lumpectomy with radiation, to lumpectomy with a receptor targeted chemotherapy, to lumpectomy with radiation with a receptor targeted chemotherapy, to mastectomy, to mastectomy with a receptor targeted chemotherapy, to mastectomy with radiation, to mastectomy with radiation and a receptor targeted chemotherapy, to surgery with a chemotherapy. In some embodiments, a subject at risk of DCIS recurrence, but not invasive breast cancer can receive adjuvant DCIS treatment (optionally, in combination with any of the embodiments provided herein).

A “marker” refers to a measured biological component such as an mRNA transcript, or a level of DNA amplification.

The term “control” refers to a sample or standard used for comparison with a sample which is being examined, processed, characterized, analyzed, etc. In some embodiments, the control is a sample obtained from a healthy patient or a non-tumor tissue sample obtained from a patient diagnosed with a breast tumor. In some embodiments, the control is a historical control or standard reference value or range of values (such as a previously tested control sample, such as a group of breast tumor patients with poor prognosis, or group of samples that represent baseline or normal values, such as the level of cancer-associated genes in non-tumor tissue).

The “Cox hazard ratio” is derived from the Cox proportional hazards model. Proportional hazards models are a class of survival models in statistics. Survival models relate the time that passes before some event occurs to one or more covariates that may be associated with that quantity of time. In the Cox proportional hazards model, the unique effect of a unit increase in a covariate is multiplicative with respect to the hazard rate. A “Cox hazard ratio” is the ratio of the hazard rates corresponding to the conditions described by two levels of an explanatory variable—a covariate, that is calculated using the cox proportional hazards model. The cox hazard ratio is the ratio of survival hazards for a one-unit change in the covariate. For example, the Cox hazard ratio may be the ratio of survival hazards for a 1 unit change in the logarithmic gene expression level. Thus, a larger value has a greater effect on survival or the hazard rate of the event being assessed, such as disease recurrence. In some embodiments, a hazard ratio (HR) greater than 1 indicates that an increased covariate level is associated with a worse patient outcome, where the covariate level is a marker expression level. In some embodiments, a HR less than 1 indicates that a decreased covariate level is associated with a better patient outcome, where the covariate level is a marker expression level.

As used herein, the term “non-tumor tissue sample” shall be taken to include any sample from or including a normal or healthy cell or tissue, or a data set produced using information from a normal or healthy cell or tissue. For example, the non-tumor sample may be selected from the group comprising or consisting of: (i) a sample comprising a non-tumor cell; (ii) a sample from a normal tissue; (iii) a sample from a healthy tissue; (iv) an extract of any one of (i) to (iii); (v) a data set comprising measurements of modified chromatin and/or gene expression for a healthy individual or a population of healthy individuals; (vi) a data set comprising measurements of modified chromatin and/or gene expression for a normal individual or a population of normal individuals; and (vii) a data set comprising measurements of the modified chromatin and/or gene expression from the subject being tested wherein the measurements are determined in a matched sample having normal cells. Preferably, the non-tumor sample is (i) or (ii) or (v) or (vii).

As used herein, the term “subject” encompasses any animal including humans, preferably a mammal. Exemplary subjects include but are not limited to humans, primates, livestock (e.g. sheep, cows, horses, donkeys, pigs), companion animals (e.g. dogs, cats), laboratory test animals (e.g. mice, rabbits, rats, guinea pigs, hamsters), captive wild animals (e.g. fox, deer). Preferably the mammal is a human or primate. More preferably the mammal is a human.

Detecting expression of a gene product denotes determining of a level expression in either a qualitative or quantitative manner can detect nucleic acid molecules. Exemplary methods include, but are not limited to: microarray analysis, RT-PCR, Northern blot, Western blot, next generation sequencing, and mass spectrometry.

The term “diagnosis” denotes the process of identifying a disease by its signs, symptoms and results of various tests. The conclusion reached through that process is also called “a diagnosis.” Forms of testing commonly performed include biopsy for the collection of the tumor. In some embodiments, the prognosis can be a high or low likelihood of a subsequent (within the next 10 years, 15, or 20 years) invasive breast cancer event.

“Differential or alteration in expression” denotes a difference or change, such as an increase or decrease, in the amount of RNA [symbol] In some examples, the difference is relative to a control or reference value or range of values, such as an amount of gene expression that is expected in a subject who does not have an invasive breast cancer or in non-tumor tissue from a subject with a breast tumor. Detecting differential expression can include measuring a change in gene expression.

The term “expression” denotes the process by which the coded information of a gene is converted into an operational, non-operational, or structural part of a cell, such as the synthesis of an RNA. Gene expression can be influenced by external signals. For instance, exposure of a cell to a hormone may stimulate expression of a hormone-induced gene. Different types of cells can respond differently to an identical signal. Expression of a gene also can be regulated anywhere in the pathway from DNA to RNA. Regulation can include controls on transcription, translation, RNA transport and processing, degradation of intermediary molecules such as mRNA, or through activation, inactivation, compartmentalization.

The expression of a nucleic acid molecule in a sample can be altered relative to a control sample, such as a normal or non-tumor sample. Alterations in gene expression, such as differential expression, include but are not limited to: (1) overexpression; (2) underexpression; or (3) suppression of expression.

Controls or standards for comparison to a sample, for the determination of differential expression, include samples believed to be normal (in that they are not altered for the desired characteristic, for example a sample from a subject who does not have invasive breast cancer in the 10 years following the event, as well as laboratory values (e.g., range of values), even though possibly arbitrarily set, keeping in mind that such values can vary from laboratory to laboratory. Laboratory standards and values can be set based on a known or determined population value and can be supplied in the format of a graph or table that permits comparison of measured, experimentally determined values. In some embodiments, the controls can be standardized levels set by housekeeping genes, as shown in table 2.

As will be appreciated by one of skill in the art, any of the above controls or standards can be provided for any of the methods (such as treatment, analysis, or prognosis) provided herein, and for any of the compositions or methods. These can be positive or negative controls or standards (showing, for example, what a high level or normal level of expression or presence of the molecule is). The controls can be matched for the relevant molecule type as well (e.g., RNA). In some embodiments, the control and/or standard can be for PD-1.

The phrase “gene expression profile” (or signature) denotes a differential or altered gene expression that can be detected by changes in the detectable amount of gene expression (such as cDNA, mRNA) [symbol] A distinct or identifiable pattern of gene expression, for instance a pattern of high and low expression of a defined set of genes or gene-indicative nucleic acids such as ESTs. In some examples, as few as one gene provides a profile, but more genes can be used in a profile, for example, at least 2, 3, 4, 5, 6, or 7 markers (e.g., genes) can be employed to provide a prediction as to the effectiveness of a particular therapy. Gene expression profiles can include relative as well as absolute expression levels of specific genes, and can be viewed in the context of a test sample compared to a baseline or control sample profile (such as a sample from the same tissue type from a subject who does not have a tumor). In some embodiments, a gene expression profile in a subject is read on an array (such as a nucleic acid). For example, a gene expression profile can be performed using a commercially available array such as Human Genome GeneChip™ arrays from Affymetrix™ (Santa Clara, Calif.). In some embodiments, any two or more of the markers indicated herein (including PD-1 and other markers or controls, such as the housekeeping genes in Table 2) can be employed as a profile or part of a profile analysis.

The term “hybridization” means to form base pairs between complementary regions of two strands of DNA, RNA, or between DNA and RNA, thereby forming a duplex molecule, for example. Hybridization conditions resulting in particular degrees of stringency will vary depending upon the nature of the hybridization method and the composition and length of the hybridizing nucleic acid sequences. Generally, the temperature of hybridization and the ionic strength (such as the sodium concentration) of the hybridization buffer will determine the stringency of hybridization. Calculations regarding hybridization conditions for attaining particular degrees of stringency are discussed in Sambrook et al., (1989) Molecular Cloning, second edition, Cold Spring Harbor Laboratory, Plainview, N.Y. (chapters 9 and 11).

The term “isolated” as used in an “isolated” biological component (such as a nucleic acid molecule, protein, or cell) is one that has been substantially separated or purified away from other biological components in the cell of the organism, or the organism itself, in which the component naturally occurs, such as other chromosomal and extra-chromosomal DNA and RNA, proteins and cells. Nucleic acid molecules and proteins that have been “isolated” include nucleic acid molecules and proteins purified by standard purification methods. The term also embraces nucleic acid molecules and proteins prepared by recombinant expression in a host cell as well as chemically synthesized nucleic acid molecules and proteins. In some embodiments, an isolated cell is an invasive breast cancer cell that is substantially separated from other breast cell types, such as non-tumor breast cells.

The term “label” or “probe” denotes an agent capable of detection, for example by ELISA, spectrophotometry, flow cytometry, or microscopy. For example, a label can be attached to a nucleic acid molecule or protein (such as one that can hybridize or bind to any of the markers provided herein (including PD-1)), thereby permitting detection of the nucleic acid molecule or protein. Examples of labels include, but are not limited to, radioactive isotopes, enzyme substrates, co-factors, ligands, chemiluminescent agents, fluorophores, haptens, enzymes, and combinations thereof. Methods for labeling and guidance in the choice of labels appropriate for various purposes are discussed for example in Sambrook et al. (Molecular Cloning: A Laboratory Manual, Cold Spring Harbor, N.Y., 1989) and Ausubel et al. (In Current Protocols in Molecular Biology, John Wiley & Sons, New York, 1998). In some embodiments, a label is conjugated to a binding agent that specifically binds to PD-1 to allow for detecting the presence of the marker in a subject or a sample from the subject.

The term “mammal” includes both human and non-human mammals. Examples of mammals include, but are not limited to: humans, pigs, cows, goats, cats, dogs, rabbits, rats, and mice.

A nucleic acid array is an arrangement of nucleic acids (such as DNA or RNA) in assigned locations on a matrix, such as that found in cDNA arrays, or oligonucleotide arrays.

A “nucleic acid molecules representing genes” is any nucleic acid, for example DNA (intron or exon or both), cDNA, or RNA (such as mRNA), of any length suitable for use as a probe or other indicator molecule, and that is informative about the corresponding gene.

“Polymerase chain reaction” (PCR) is an in vitro amplification technique that increases the number of copies of a nucleic acid molecule (for example, a nucleic acid molecule in a sample or specimen), such as amplification of a nucleic acid molecule for PD-1. The product of a PCR can be characterized by standard techniques known in the art, such as electrophoresis, restriction endonuclease cleavage patterns, oligonucleotide hybridization or ligation, and/or nucleic acid sequencing. In some examples, PCR utilizes primers, for example, DNA oligonucleotides 10-100 nucleotides in length, such as about 15, 20, 25, 30 or 50 nucleotides or more in length (such as primers that can be annealed to a complementary target DNA strand by nucleic acid hybridization to form a hybrid between the primer and the target DNA strand, such as PD-1). Primers can be selected that include at least 15, at least 20, at least 25, at least 30, at least 35, at least 40, at least 45, at least 50 or more consecutive nucleotides of a marker provided herein. Methods for preparing and using nucleic acid primers are described, for example, in Sambrook et al. (In Molecular Cloning: A Laboratory Manual, CSHL, New York, 1989), Ausubel et al. (ed.) (In Current Protocols in Molecular Biology, John Wiley & Sons, New York, 1998), and Innis et al. (PCR Protocols, A Guide to Methods and Applications, Academic Press, Inc., San Diego, Calif, 1990).

The term “prognosis” denotes a prediction of the course of a disease. In some embodiments provided herein, the phrase, when used in the context of a person already having invasive breast cancer, denotes the likelihood that a subject having the invasive breast cancer will go on (within a following ten, fifteen, or twenty year period) to have a subsequent ipsilateral invasive breast cancer event after surgical removal of the primary tumor. The prediction can include determining a) the likelihood of an ipsilateral breast event, b) the likelihood of an ipsilateral breast event in a particular amount of time (e.g., 1, 2, 3 or 5 years), c) the likelihood that a particular therapy (e.g., radiation) will prevent an ipsilateral breast event, d) an optimal treatment to help prevent an ipsilateral event that matches the severity of the most likely event, or e) combinations thereof.

The phrase “specific binding agent” denotes an agent that binds substantially or preferentially only to a defined target such as a protein, enzyme, polysaccharide, oligonucleotide, DNA, RNA, recombinant vector or a small molecule. In an example, a “specific binding agent” is capable of binding to at least one of the disclosed markers (such as PD-1). In some embodiments, the specific binding agent is capable of binding to a downstream factor regulated by at least one of the disclosed markers (such as PD-1). Thus, a nucleic acid-specific binding agent binds substantially only to the defined nucleic acid, such as RNA, or to a specific region within the nucleic acid. For example, a “specific binding agent” includes an antisense compound (such as an antisense oligonucleotide, siRNA, miRNA, shRNA or ribozyme) that binds substantially to a specified RNA.

The term “radiation therapy” denotes a therapy that involves or includes some form of radiation in an amount that is therapeutic to the subject.

The term “standard radiation therapy” denotes a therapy that involves or includes some form of radiation in an amount that is therapeutic to the subject under the current standard of care for breast cancer. In some embodiments, the standard of care is any one that is provided in NCCN, ESMO, Clinical Practice Recommendations Australia, or NICE guideline, and optionally, any one or more of the respective guidelines as of May of 2021. In some embodiments, the standard of case is any one of the following in table 3 below.

TABLE 3

| | N- | | | | |
|---|---|---|---|---|---|
| Guideline | status | Surgery | Volume | Fractionation | Boost (tumor bed) |
| NCCN | NC | BCS | WBRT * and | 40-42.5 Gy in 15-16 | 10-16 Gy in 4-8 fraction |
| | | | ** | fractions (or 45-50.5 Gy | if high risk *** |
| | | | | in 25-28 Fr.) | |
| NCCN | N+ | BCS | WBRT +RNI | 40-42.5 Gy in 15-16 | 10-16 Gy in 4-8 fraction |
| | | | | fractions (or 45-50.5 Gy | if high risk *** |
| | | | | in 25-28 Fr.) | |
| ESMO | NO | BCS | WBRTS | 45-50 Gy in 25-28 | 10-16 Gy in 4-8 fraction |
| | | | | fractions (or 2.5 -2.67 Gy | if high risk *** |
| | | | | X 15-16 Fr) | |
| ESMO | N+ | BCS | WBRT+RNI | 45-50 Gy in 25-28 | 10-16 Gy in 4-8 fraction |
| | | | | fractions (or 2.5 -2.67 Gy | if high risk *** |
| | | | | X 15-16 Fr) | |
| NICE | NO | BCS | WBRT § | 40 Gy in 15 fractions | 10 Gy in 5 fractions if |
| | | | | | high risk z |
| NICE | N+ | BCS | WBRT+RNI | 40 Gy in 15 fractions | 10 Gy in 5 fractions if |
| | | | | | high risk z |

NCCN Guidelines Version 4.2021 (Invasive Breast Cancer)
PRINCIPLES OF RADIATION THERAPY
Optimizing Delivery of Individual Therapy
It is important to individualize RT planning and delivery.
CT-based treatment planning should be routinely utilized to delineate target volumes and
adjacent organs at risk.
Radiation to the breast/chest wall and nodal regions is generally delivered with single energy
or mixed energy photonst electrons.
Improved homogeneity of the target dose and sparing of normal tissues can be
accomplished using compensators such as wedges, forward planning using segments, and
intensity-modulated RT (IMRT).
Additional techniques such as respiratory control (deep inspiration breath-hold), prone
positioning, cardiac blocks may also be used to try to further reduce dose to heart, lung, and
adjacent normal tissue.
Verification of treatment setup consistency is done with weekly imaging. When using certain
techniques (ie, prone breast), more frequent imaging may be appropriate. Standard
utilization of daily imaging is not recommended.
When treating the internal mammary nodes, dose-volume histograms (DVHs) should be
used to evaluate dose constraints, dose to normal tissues (ie, heart, lung), and planning
target volumes (PTVs).
It is common for RT to follow chemotherapy when chemotherapy is indicated.
Whole Breast Radiation
Target definition is the breast tissue in entirety.
RT dosing:
The whole breast should receive a hypofractionated dose of 40-42.5 Gy in 15-16 fractions; in
selected cases 45-50.4 Gy in 25-28 fractions may be considered.
A boost to the tumor bed is recommended in patients at higher risk for recurrence. Typical
boost doses are 10-16 Gy in 4-8 fractions.
Lumpectomy cavity boost can bedelivered using enface electrons, photons, or
brachytherapy.
For patients who require a more limited number of treatment visits for WBRT delivery,
ultra-hypofractionated WBRT of 28.5 Gy delivered as 5 (once-a-week) fractions, may be
considered in selected patients aged ≥50 years following BCS with pTis/T1/T2/NO tumor.
However, late toxicity effects beyond 10 years are not currently defined.
The optimal fractionation for the delivery of a boost is not known for this regimen.
3-D planning o minimize inhomogeneity and exposure to heart and lung is essential when
using this regimen.
ESMO guidelines
RadiotWhole-breastherapy.
RT after BCS: Postoperative RT is strongly rec- ommended after BCS [I, A]. WBRT alone reduces
the 10-year risk of any first recurrence (including locoregional and distant) by 15% and the 15-year risk
of breast cancer-related mortality by 4%. Boost RT gives a further 50% RR reduction and is indi- cated
for most patients who have unfavourable risk factors for local control such as age <50 years, grade 3
tumours, presence of vascular invasion or extensive intraductal component and non- radical tumour
excision (focally-otherwise further surgery should be advocated) [I, A].
Recommendations:
Postoperative RT is strongly recommended after BCS [I, A].
Boost RT is recommended to reduce the risk of in-breast re-
lapse in patients at higher risk of local recurrence [I, A].
Accelerated partial-breast RT after BCS: The concept of accelerated partial-breast irradiation
(APBI) is an appealing approach to substantially shorten the overall treatment time. The rationale for
APBI is that the majority of local failures occur in the vicinity of the primary tumour site, while so-called
'elsewhere' in-breast failures may represent a new primary tumour. Excellent results with low local
recurrence rates equivalent to WBRT are reported for partial-breast irradiation (accelerated and
conventionally fractionated) using external beam techniques and brachytherapy. However, for
intraoperative RT, as used in the ELIOT (single dose with electrons) and TARGIT (single dose with 50-kV
X- rays) randomised trials, the ipsilateral breast cancer recurrence rate was significantly higher in the
APBI groups, compared with the WBRT. Based on these results, APBI might be considered an
acceptable treatment option in patients with a low risk for local recurrence, for example those who are
at least 50years old, with unicentric, unifocal, node-negative, non- lobular breast cancer, up to 3 cm
without the presence of extensive intraductal components or vascular invasion and with negative
margins, especially if they will receive adjuvant endocrine treatment [III, C]. APBI may also be
considered for low-grade DCIS [III, C]. More and long-term results of several past and ongoing
prospective randomised APBI trials are awaited.
Recommendation:
APBI is an acceptable treatment option in patients with a low risk for local recurrence [III, C].
Early and locally advanced breast cancer: diagnosis and management
NICE guideline [NG101] Published: 18 July 2018
Radiotherapy after breast-conserving surgery
1.10.3 Offer whole-breast radiotherapy to women with invasive breast cancer who have had
breast-conserving surgery with clear margins. [2018]
1.10.4 Consider partial breast radiotherapy (as an alternative to whole-breast radiotherapy) for women
who have had breast-conserving surgery for invasive cancer (excluding lobular type) with clear
margins and who:
have a low absolute risk of local recurrence (defined as women aged 50 and over with tumours
that are 3 cm or less, NO, ER-positive, HER2-negative and grade 1 to 2) and
have been advised to have adjuvant endocrine therapy for a minimum of 5 years. [2018]
1.10.5 When considering partial breast radiotherapy (see recommendation 1.10.4), discuss the benefits
and risks, and explain that:
local recurrence with partial breast radiotherapy at 5 years is equivalent to that with
whole-breast radiotherapy
the risk of local recurrence beyond 5 years is not yet known
there is a potential reduction in late adverse effects. [2018]
1.10.6 When delivering partial breast radiotherapy, use external beam radiotherapy. [2018]
1.10.7 Consider omitting radiotherapy for women who:
have had breast-conserving surgery for invasive breast cancer with clear margins and
have a very low absolute risk of local recurrence (defined as women aged 65 and over with
tumours that are T1NO, ER-positive, HER2-negative and grade 1 to 2) and
are willing to take adjuvant endocrine therapy for a minimum of 5 years. [2018]
1.10.8 When considering omitting radiotherapy for the population in recommendation 1.10.7, discuss
the benefits and risks ... and explain that:
without radiotherapy, local recurrence occurs in about 50 women per 1,000 at 5 years, and
with radiotherapy, occurs in about 10 women per 1,000 at 5 years
overall survival at 10 years is the same with or without radiotherapy
there is no increase in serious late effects if radiotherapy is given (for example, congestive
cardiac failure, myocardial infarction or secondary cancer). [2018]
Dose fractionation
1.10.13 Use external beam radiotherapy giving 40 Gy in 15 fractions as standard practice for
women with invasive breast cancer after breast-conserving surgery or mastectomy. [2009]
Breast boost following breast-conserving surgery
1.10.14 Offer an external beam boost to the tumour bed for women with invasive breast cancer
and a high risk of local recurrence, following whole-breast radiotherapy. [2009, amended 2018]
1.10.15 Inform women of the risk of side effects associated with an external beam boost to the
tumour bed following whole-breast radiotherapy. [2009, amended 2018]
Radiotherapy to nodal areas
1.10.16 Do not offer adjuvant radiotherapy to regional lymph nodes to people with invasive breast
cancer who have been shown to have histologically lymph node-negative breast cancer. [2009,
amended 2018]
1.10.17 Do not offer adjuvant radiotherapy to the axilla after axillary clearance for invasive breast
cancer. [2009, amended 2018]
1.10.18 Offer adjuvant radiotherapy to the supraclavicular fossa to people with invasive breast
cancer and 4 or more involved axillary lymph nodes. [2009]
1.10.19 Offer adjuvant radiotherapy to the supraclavicular fossa to people with invasive breast
cancer and 1 to 3 positive lymph nodes if they have other poor prognostic factors (for example, T3
and/or histological grade 3 tumours) and good performance status. [2009]
1.10.20 Consider including the internal mammary chain within the nodal radiotherapy target for
people with node-positive (macrometastases) invasive breast cancer. [2018]
International guidelines
The following international guidelines have been identified which include guidance on the use
of hypofractionated radiotherapy for early breast cancer:
The American Society for Radiation Oncology (ASTRO) guidelines on fractionation for whole
breast irradiation, 2010
The New Zealand Ministry of Health Guidelines for Management of Early Breast Cancer, 2009
NICE Guidelines for early and locally advanced breast cancer, 2009
Scottish Intercollegiate Guidelines Network (SIGN) guidelines, 2009
BC Cancer Agency Breast cancer management consensus guidelines 2013
European Journal of Medical Oncology (ESMO) guidelines on primary breast cancer diagnosis,
treatment and follow-up, 2013
German Society of Radiation Oncology (DEGRO) guidelines on radiotherapy of breast cancer,
2013
Nice-Saint-Paul de Vence guidelines on adjuvant radiotherapy in the management of axillary
node negative invasive breast cancer, 2013
The BC Cancer Agency consensus based guidelines for the management of early breast cancer
include recommendations on the use of radiotherapy and recommend a hypofractionated radiotherapy
regimen as standard. The guideline recommends the following dose fractionation for radiotherapy
following breast conserving therapy (T1, T2; NO):
1. Standard whole breast dose is 42.5 Gray (Gy) in 16 daily fractions
2. Certain patients are at risk for inferior cosmetic outcome from the 16-fraction course.
Extended fractionation should be considered for patients with very large breast size, and those
with significant post-operative induration, oedema, erythema, hematoma or infection. Patients
with these indications for extended fractionation should receive 45Gy in 25 daily fractions plus
a boost dose of 10Gy in 5 fractions or 50.4 Gy in 28 daily fractions.
3. If a boost is used, an additional dose of 6-16 Gy in 3-8 fractions is recommended.
WBRT = Whole breast radiotherapy
RNI = regional nodal irradiation
BCS = breast-conserving surgery
NO = no lymph node involvement
N+ = lymph node involvement
APBI if low risk
** omission considered if >70 ER+, T1, Endocrine Treatment (ET)
*** >2 cm, younger age, LVI (lymphovascular invasion)
§ APBI if age < 50, T <= 3, ER-positive, HER2-negative and grade 1 to 2, non-lobular
z Risk can be estimated using a range of standardised tools and clinical expertise

The term “non-radiation therapy” denotes a therapy that is adequate for addressing or reducing the risk of invasive breast cancer in a subject, and that does not derive its therapeutic effect by radiation. Examples of such therapy include, chemo therapeutics, targeted and non targeted, immune and non-immune modulated, monoclonal, other targeted and non-targeted, genomic therapies, antibody therapeutics, including, HER2 antibodies, including Trastuzumab. Often, in the present application, “non-radiation therapy” is denoted as “other therapy”.

The term “Local recurrence” denotes that a recurrence is in the operated breast.

The term “Regional recurrence” denotes that a recurrence is in regional lymph nodes (axillary, supraclavicular, infraclavicular, intrapectoral or internal mammary lymph nodes).

The term “distant metastasis” refers to all other recurrences outside the above types of recurrences (local or regional). In other words, distant metastasis refers to recurrences in all other tissues of the body.

In some embodiments, the methods provided herein are not applied to distant metastasis. In some embodiments, the methods provided herein are applied to local, regional, and/or local and regional recurrences.

As noted above, provided herein are methods for determining a likelihood that a subject will benefit from radiation therapy and then providing a selected therapy to the subject according to that, and optionally other, results. Subjects with a PD-1 level above a specific threshold are those that are most likely to benefit from standard or the current standard of care for radiation therapy. Those with PD-1 level below the threshold are those who will benefit from an alternative therapy (such as non-radiation or elevated radiation levels).

Some embodiments provided herein relate to a method for treating breast cancer (both invasive and in situ). The method comprises the steps: a) obtaining a tissue sample of a tumour from a breast cancer patient, b) determining the expression levels of PD-1 mRNA in the sample, c) determining that the expression level is below a threshold level, d) providing intensified treatment as intensified radiotherapy treatment, intensified systemic therapy or mastectomy to the patient.

In some embodiments, the threshold of a PD-1 mRNA below the 25^{th} percentile of a breast cancer reference population. In some embodiments, PD-1 mRNA expression can be defined as the levels of mRNA transcripts of the PD-1 gene (PDCD1). In some embodiments, absolute levels can be measured by RNA seq or PCR for example. In some embodiments, a relative expression level can be used, e.g., by using a microarray and comparing the level to the levels in a standard population or sample. In some embodiments, the reference standard can be that in the Gene Expression Omnibus library with GEO accession number GSE119295.

In some embodiment, the cancer patient can have invasive breast cancer. In some embodiment, the method can also be used for ductal carcinoma in situ (DCIS). In some embodiments, the patient may be a patient that has undergone surgery, which preferably is breast-conserving surgery or mastectomy, where breast-conserving surgery is preferred. The patient is preferably a female patient in the case of breast cancer. In some embodiments, the patients can have undergone axillary lymph node dissection. In some embodiments, the patients have not undergone axillary lymph node dissection. In some embodiments, sentinel node biopsy has been performed on the patient.

In some embodiment, the patient has been diagnosed with early stage breast cancer. In some embodiments, the early stage breast cancer may be lymph node negative (NO) stage 0 (ductal cancer in situ), I or IIA cancer. The stage is determined as in known in the art of pathology. Thus, it is preferred that the tumor has a diameter less than 5 cm, and there are no macrometastases in the lymph nodes.

In some embodiments, the subject has a tumor that has a tumor stage of T1T2N0M0. In some embodiments, the subject has a tumor that has a tumor stage of T1T2N1M0.

In some embodiment, a sample is taken from the tumor of the patient. The sample may be a biopsy which is taken before surgery or during surgery. The sample may be a breast cancer tissue biopsy. The sample can be a biopsy from the operated tumor but other biopsy alternatives include core biopsy, excisional biopsy, stereotactic biopsy and fine-needle aspiration. The biopsy may comprise CD8+cells, which is the cell type that typically expresses PD-1. Hence, the biopsy may comprise T-cells which have infiltrated the tumour. The sample may comprise circulating tumour cells.

When the sample has been collected, it can be treated in a variety of ways. In some embodiments, gene expression profiling is performed on fresh frozen or formalin-fixed paraffin-embedded tissue. When an antibody is used for visually detecting PD-1 the sample can be paraffin embedded.

In some embodiments, the expression level of PD-1 mRNA in the sample is determined. Detection of PD-1 expression may be carried out using any suitable method that makes it possible to quantify expression level of PD-1. Various suitable methods may be used to detect PD-1 expression. PD-1 expression determination may be carried out at the mRNA level. PD-1 expression may for example be detected using northern blot, quantitative PCR, whole RNA sequencing, expression arrays, in situ hybridization. A useful guide to various techniques useful in the detection of macromolecules is Current Protocols in Molecular Biology, Current Protocols in Human Genetics, and current protocols in Immunology, all published by the Wiley group.

In some embodiments, sequencing methods can be used to determine the levels of PD-1 mRNA in the sample. Sequencing methods may include Sanger sequencing or next generation sequencing (NGS) methods. For example, total mRNA of the sample may be sequenced, for example using NGS, and the number of sequence reads is counted, where the number or sequence reads correlate with levels of PD-1 mRNA in the sample.

In some embodiments, the expression level can be in relation to a reference group, where the PD-1 expression level of the members of the group has been quantified and the PD-1 expression level of the patient is compared to the group. The reference group may be a group of patients that is similar to the patient being treated (for example women with early breast cancer). The expression level in the reference group is preferably determined using the same method as for the patient. Typically, each pathology laboratory will have their own reference group. In some embodiments, the reference standard can be that of the Gene Expression Omnibus library with GEO accession number GSE119295.

In some embodiments, the expression levels of the reference group are ranked according to PD-1 expression level and a threshold expression level is determined as the expression level of a predetermined percentile of the reference group. For example, the threshold can be determined as the expression level of the 25^{th} percentile, where expression levels over the 25^{th} percentile are considered to be high expression. In some embodiments, other cut-offs may be used, such as less than 50^{th}, the 10^{th}-40^{th}, or 10^{th}-30^{th} percentile. Hence the cut-off may be for example the 15^{th}, 20^{th}, the 25^{th} or the 30^{th} or the 40^{th} percentile. The number of patients in the reference group is preferably at least 100, more preferably at least 1000. In some embodiments, the reference group is a grouping within the Gene Expression Omnibus library with GEO accession number GSE119295.

In some embodiments, the mRNA transcript of PD-1 is detected.

In some embodiments, it is determined that the expression level of PD-1 of a cancer patient is below a threshold level and intensified treatment is provided to the cancer patient. In some embodiments, the intensified treatment can comprise intensified, elevated, or aggressive radiotherapy treatment is administered to the subject. In some embodiments, this can instead be (or include) intensified systemic therapy or mastectomy.

In some embodiments, the intensified or aggressive or elevated radiotherapy treatment is one or more of whole breast external radiotherapy, partial breast radiotherapy or brachytherapy or a combination thereof, with a biologically effective dose of (BED) of 73 Gy or more with a tumor alpha/beta ratio of 5 or a BED of 78 Gy or more with a tumor alpha/beta ratio of 4 or a BED of 87 Gy or more with a tumor alpha/beta ratio of 3 or a BED of 104 Gy or more with a tumor alpha/beta ratio of 2 for patients who are not otherwise recommended a boost dose according to the current guidelines.

In some embodiments, intensified or aggressive or elevated radiotherapy treatment is one or more of whole breast external radiotherapy, partial breast radiotherapy or brachytherapy or a combination thereof, with a biologically effective dose of (BED) of 93 Gy or more with a tumor alpha/beta ratio of 5 or a BED of 100 Gy or more with a tumor alpha/beta ratio of 4 or a BED of 111 Gy or more with a tumor alpha/beta ratio of 3 or a BED of 133 Gy or more with a tumor alpha/beta ratio of 2 for patients who are recommended a boost according to the current guidelines. In some embodiments, BED is as a measure of the true biological dose delivered by a combination of dose per fraction (d) and number of fractions (n) to a tissue characterized by a specific radiosensitivity (alfa/beta ratio): BED=nd[1+d/(alfa/beta)].

In some embodiments, the patient has been subjected to breast conserving surgery or total mastectomy. Thus, in some embodiments, any of the methods provided herein can involve or be applied to a subject who has already gone through breast conserving surgery or total mastectomy.

In some embodiments, the breast cancer that the subject currently has is an early stage invasive breast cancer or breast cancer in situ.

In some embodiments, the expression level of PD-1 is determined by detecting the amount of PD-1 mRNA in the sample.

In some embodiments, the PD-1 mRNA comprises the nucleotide sequence of SEQ ID NO 1, below. It is to be noted that the sequence below is described using Ts instead of U:s because it is from a cDNA. The actual mRNA has uracil instead of thymidines. In some embodiments, any probe that hybridizes to the mRNA PD-1 below can be used. In some embodiments, any probe that is 6-30 nucleotides in length, and is at least 80% complementary to 6-30 nucleotides of SEQ ID NO:1 can be used, for example any 6-30 nucleic acid sequence that is 85, 90, 95, or 100% identical to any 6-30 contiguous sequence in SEQ ID NO: 1 can be used.

PD-1 mRNA:
(SEQ ID NO 1)
GCTCACCTCCGCCTGAGCAGTGGAGAAGGCGGCACTCTGGT
GGGGCTGCTCCAGGCATGCAGATCCCACAGGCGCCCTGGC
CAGTCGTCTGGGCGGTGCTACAACTGGGCTGGCGGCCAGG
ATGGTTCTTAGACTCCCCAGACAGGCCCTGGAACCCCCCC
ACCTTCTCCCCAGCCCTGCTCGTGGTGACCGAAGGGGACA
ACGCCACCTTCACCTGCAGCTTCTCCAACACATCGGAGAG
CTTCGTGCTAAACTGGTACCGCATGAGCCCCAGCAACCAG
ACGGACAAGCTGGCCGCCTTCCCCGAGGACCGCAGCCAGC
CCGGCCAGGACTGCCGCTTCCGTGTCACACAACTGCCCAA
CGGGCGTGACTTCCACATGAGCGTGGTCAGGGCCCGGCGC
AATGACAGCGGCACCTACCTCTGTGGGGCCATCTCCCTGG
CCCCCAAGGCGCAGATCAAAGAGAGCCTGCGGGCAGAGCT
CAGGGTGACAGAGAGAAGGGCAGAAGTGCCCACAGCCCAC
CCCAGCCCCTCACCCAGGCCAGCCGGCCAGTTCCAAACCC
TGGTGGTTGGTGTCGTGGGCGGCCTGCTGGGCAGCCTGGT
GCTGCTAGTCTGGGTCCTGGCCGTCATCTGCTCCCGGGCC
GCACGAGGGACAATAGGAGCCAGGCGCACCGGCCAGCCCC
TGAAGGAGGACCCCTCAGCCGTGCCTGTGTTCTCTGTGGA
CTATGGGGAGCTGGATTTCCAGTGGCGAGAGAAGACCCCG
GAGCCCCCCGTGCCCTGTGTCCCTGAGCAGACGGAGTATG
CCACCATTGTCTTTCCTAGCGGAATGGGCACCTCATCCCC
CGCCCGCAGGGGCTCAGCTGACGGCCCTCGGAGTGCCCAG
CCACTGAGGCCTGAGGATGGACACTGCTCTTGGCCCCTCT
GACCGGCTTCCTTGGCCACCAGTGTTCTGCAGACCCTCCA
CCATGAGCCCGGGTCAGCGCATTTCCTCAGGAGAAGCAGG
CAGGGTGCAGGCCATTGCAGGCCGTCCAGGGGCTGAGCTG
CCTGGGGGCGACCGGGGCTCCAGCCTGCACCTGCACCAGG
CACAGCCCCACCACAGGACTCATGTCTCAATGCCCACAGT
GAGCCCAGGCAGCAGGTGTCACCGTCCCCTACAGGGAGGG
CCAGATGCAGTCACTGCTTCAGGTCCTGCCAGCACAGAGC
TGCCTGCGTCCAGCTCCCTGAATCTCTGCTGCTGCTGCTG
CTGCTGCTGCTGCTGCCTGCGGCCCGGGGCTGAAGGCGCC
GTGGCCCTGCCTGACGCCCCGGAGCCTCCTGCCTGAACTT
GGGGGCTGGTTGGAGATGGCCTTGGAGCAGCCAAGGTGCC
CCTGGCAGTGGCATCCCGAAACGCCCTGGACGCAGGGCCC
AAGACTGGGCACAGGAGTGGGAGGTACATGGGGCTGGGGA
CTCCCCAGGAGTTATCTGCTCCCTGCAGGCCTAGAGAAGT
TTCAGGGAAGGTCAGAAGAGCTCCTGGCTGTGGTGGGCAG
GGCAGGAAACCCCTCCACCTTTACACATGCCCAGGCAGCA
CCTCAGGCCCTTTGTGGGGCAGGGAAGCTGAGGCAGTAAG
CGGGCAGGCAGAGCTGGAGGCCTTTCAGGCCCAGCCAGCA
CTCTGGCCTCCTGCCGCCGCATTCCACCCCAGCCCCTCAC
ACCACTCGGGAGAGGGACATCCTACGGTCCCAAGGTCAGG
AGGGCAGGGCTGGGGTTGACTCAGGCCCCTCCCAGCTGTG
GCCACCTGGGTGTTGGGAGGGCAGAAGTGCAGGCACCTAG
GGCCCCCCATGTGCCCACCCTGGGAGCTCTCCTTGGAACC
CATTCCTGAAATTATTTAAAGGGGTTGGCCGGGCTCCCAC
CAGGGCCTGGGTGGGAAGGTACAGGCGTTCCCCCGGGGCC
TAGTACCCCCGCCGTGGCCTATCCACTCCTCACATCCACA
CACTGCACCCCCACTCCTGGGGCAGGGCCACCAGCATCCA
GGCGGCCAGCAGGCACCTGAGTGGCTGGGACAAGGGATCC
CCCTTCCCTGTGGTTCTATTATATTATAATTATAATTAAA
TATGAGAGCATGCTAA

In some embodiments, the PD-1 mRNA can be extracted from the sample. Extraction can be done using any suitable method. For example, phenol extraction or using TRIzol reagent (ThermoFisher).

In some embodiments, the method may involve using a polynucleotide probe that is able to hybridize (Watson-crick base pair) to SEQ ID NO 1, that is, form Watson-Crick base pairs with SEQ ID NO 1. This is the case in for example array technologies, northern blot and also PCR techniques (where the probe is used to amplify the target sequence). The probe may be selected from the reverse complement sequence of SEQ ID NO 1. The polynucleotide probe is preferably specific for SEQ ID NO 1. The polynucleotide probe preferably has a length of at least 15 nucleotides, more preferably at least 18 nucleotides and even more preferred at least 20 nucleotides. The nucleotide is preferably able to hybridize to SEQ ID NO 1 in a specific manner, and preferably with high affinity under what the skilled person refers to as stringent conditions. When designing the polynucleotide probe it may be useful to BLAST it against other mRNA sequences that may be present in the sample such as human mRNA sequences and virus mRNA sequences.

Synthesis of polynucleotides are known in the art of organic chemistry. In general, a polynucleotide may be synthesized using suitable chemistry known in the art, where the individual nucleotides are added one by one. For example, a solid phase may be used. Of course, typically the polynucleotide probe is ordered from a company which specializes in oligonucleotide synthesis.

In some embodiments, PD-1 expression may be determined using quantitative PCR (qPCR), such as for example real-time PCR. One primer is labelled to enable detection of the PCR products. In some embodiments, a suitable system for quantitative PCR is the TaqMan system (Applied Biosystems/ThermoFisher).

Some embodiments relate to a PD-1 mRNA-binding nucleotide for use in the diagnosis of breast cancer, where the nucleotide is used for quantifying the level of PD-1 that is expressed in a breast cancer sample, and where low expression of PD-1 indicates that the patients belong to a patient subgroup where intensified radiotherapy treatment is needed.

Some embodiments relate to a method of diagnosis; the method comprises the steps of a) obtaining a tissue sample of a tumour from a breast cancer patient, b) determining the expression level of PD-1 mRNA in the sample, c) determining that the expression level is below a threshold expression level, d) thereby determining that the patient belongs to a group that would benefit from intensified radiotherapy treatment; and e) optionally providing the intensified radiotherapy treatment to the patient.

In some embodiments, the treatment can be one that is applicable to local treatment of an invasive breast cancer. In some embodiments, the subject has received a local treatment for invasive breast cancer selected from Whole-breast radiotherapy (WBRT), Standard fractionation (2 Gy fractions), Hypofractionation (2.67 Gy fractions), Fast forward (5.2 Gy fractions), Accelerated partial breast irradiation (ABPI): this is an accelerated regimen consisting of fewer fractions but with an increased frequency, Brachytherapy, Intraoperative radiotherapy, and/or External partial radiotherapy. In some embodiments, any of these can be combined with a boost which can be administered through: External radiotherapy, Brachytherapy, and/or Intraoperative radiotherapy. In some embodiments, the subject is not receiving lymph node therapy.

In some embodiments, the treatment can be one that is applicable to regional treatment of an invasive breast cancer. In some embodiments, the subject has received a regional treatment for invasive breast cancer selected from external radiotherapy: standard radiotherapy or hypofractionated radiotherapy. In some embodiments, the above-mentioned systemic therapies also decrease the risk of regional recurrences

Some embodiments relate to a method of treating a subject; the method comprises: 1) identifying an incremental risk to a subject with invasive breast cancer or in situ breast cancer of a local or regional recurrence of an invasive breast cancer based on a level of PD-1 in a sample of an invasive breast cancer in the subject; and 2) administering an intensified breast cancer therapy to the subject based upon the incremental risk, wherein a higher incremental risk will increase:
a) a likelihood of an aggressive breast cancer therapy that is at least more than what would be recommended by the NCCN;
b) the aggressiveness of the aggressive breast cancer; or
c) both a) and b).

In some embodiments, the subject is at a higher risk if they are young age at diagnosis, larger tumor size, multiple positive regional lymph nodes, high grade, high ki-67, positive HER2 status, ER negativity, high Prosigna score, high Oncotype Dx score. (Prognostic but not predictive).

A local breast cancer recurrence means that the breast cancer has come back in or near the same place it was originally found in the breast—in particular, in the remaining breast tissue of the operated breast. A local breast cancer recurrence may lead to any of the following symptoms: a new lump in the breast, a new area of the breast that seems unnaturally firm, redness or swelling of the skin in or around the breast area, flattening or other changes to the nipple, bumps on or under the skin of the chest wall, new pulling of skin or swelling at the lumpectomy site, and a new thickening on or near the mastectomy scar.

In some embodiments, an incremental risk can be identified based on a level of PD-1 in a sample of an invasive breast cancer in the subject. In other embodiments, an incremental risk can be identified based on the young age at diagnosis, larger tumor size, multiple positive regional lymph nodes, high grade, high ki-67, positive HER2 status, ER negativity, high Prosigna score, or high Oncotype Dx score.

Some embodiments relate to a method for treating a subject for recurrence of invasive breast cancer; the method comprises: 1) providing a cancer tissue sample from a subject who has invasive breast cancer; 2) analyzing the cancer tissue sample for a level of PD-1; 3) treating the subject with an intensified treatment if the cancer tissue sample has a low level of PD-1 (e.g., below the 25^{th} percentile of a reference population, such as GEO accession number GSE119295).

The expression level of PD-1 may be in relation to a reference group (such as GEO accession number GSE119295), where the PD-1 expression level of the members of the group has been quantified and the PD-1 expression level of the patient is compared to the group. In some embodiments the expression levels of members of the reference group are ranked according to PD-1 expression level and a threshold expression level is determined as the expression level of a predetermined percentile of the reference group. Expression levels below the threshold are considered to be low expression and expression levels over the threshold are considered to be high expression. In some embodiments, the threshold can be less than 50% (from e.g., GEO accession number GSE119295), such as the 10^{th}-40^{th}, more preferably the 10^{th}-30^{th} percentile.

Some embodiments relate to a method of treating a subject, the method comprising: 1) identifying a subject with invasive breast cancer that has a low level of PD-1; and 2) administering an intensified treatment to the invasive breast cancer.

Some embodiments relate to a method for recommending a treatment to a subject, said method comprising: 1) analyzing a cancer tissue sample for a level of PD-1 from a subject; 2) recommending that one treat the subject with an intensified treatment if the cancer tissue sample has a low level of PD-1 (e.g., below 50, e.g., 40, 30, 20, 10% or lower percentile of GEO accession number GSE119295); wherein the intensified is above the the current guideline, wherein the guideline can be, for example, NCCN, ESMO, Clinical Practice Recommendations Australia, or NICE guideline.

Some embodiments relate to a method for preventing an invasive breast cancer recurrence in a subject, the method comprising: 1) providing a cancer tissue sample from a subject who has invasive breast cancer; 2) analyzing the cancer tissue sample for a level of PD-1; 3) administering an intensified treatment if the cancer tissue sample has a low level of PD-1 (e.g., below 50%, 40%, 30%, or 25% percentile of GEO accession number GSE119295).

Some embodiments relate to a method for preventing an invasive breast cancer recurrence in a subject, the method comprising: receiving an intensified treatment if a cancer has a low level of PD-1 (e.g., below 25% percentile of GEO accession number GSE119295).

Some embodiments relate to a method of modifying a treatment for a subject, the method comprising: 1) identifying a subject with invasive breast cancer that has a low level of PD-1 (e.g., below 25% percentile of GEO accession number GSE119295); and 2) administering a breast cancer therapy to the subject, wherein the breast cancer therapy is more aggressive than a traditional breast cancer therapy, wherein the traditional breast cancer therapy is one recommended for the subject, based on the subject's risk factors excluding PD-1 levels.

In some embodiments, low level of PD-1 denotes a level of mRNA present in the sample. In some embodiments, low levels of PD-1 are defined by a comparison of PD-1 levels from the tissue sample to a control sample that does not include invasive cancer. In some embodiments, low are set according to the representative data in GEO accession number GSE119295, with low being less than 25, 30, 35, 40, 45, or 50% of the PD-1 levels displayed across the group.

In some embodiments, low, are determined by comparison to an internal control in the sample. In some embodiments, the control includes a cell population that has not PD-1. In some embodiments, methods described herein comprise an external control that is a cell line known to stain negative for PD-1. In some embodiments, methods described herein comprise an external control known to stain positive for PD-1 due to a high level of PD-1. In some embodiments, low levels of PD-1 are defined by a comparison to a standardized level set by a level of expression of a set of one or more housekeeping gene. In some embodiments, the housekeeping gene is one shown in Table 2 below.

TABLE 2

| Gene | | Chromo- |
|---|---|---|
| symbol | Gene name | some |
| A4galt | alpha 1,4-galactosyltransferase | chr7 |
| Actb | Actin, beta | chr12 |
| B2m | beta-2 microglobulin | chr3 |
| Cck | Cholecystokinin | chr8 |
| Cry2 | Cryptochrome 2 (photolyase-like) | chr3 |
| Csnk1g2 | Casein kinase 1, gamma 2 | chr7 |
| Decr1 | 2,4-dienoyl CoA reductase 1, mitochondrial | chr5 |
| Dimt1 | DIM1 dimethyladenosine transferase 1 | chr2 |
| | homolog (S. cerevisiae) | |
| Eeflal | Eukaryotic translation elongation factor 1 alpha 1 | chr8 |
| Farp1 | FERM, RhoGEF (Arhgef) and pleckstrin domain | chr15 |
| | protein 1 (chondrocyte-derived) | |
| Fpgs | Folylpolyglutamate synthase | chr3 |
| Gapdh | Glyceraldehyde-3-phosphate dehydrogenase | chr4 |
| Gins2 | GINS complex subunit 2 (Psf2 homolog) | chr19 |
| Gusb | Glucuronidase, beta | chr12 |
| Hmbs | Hydroxymethylbilane synthase | chr8 |
| Hprt1 | Hypoxanthine phosphoribosyltransferase 1 | chrX |
| Hsp90ab1 | Heat shock protein 90 alpha (cytosolic), class B | chr9 |
| | member 1 | |
| Mapre2 | Microtubule-associated protein, RP/EB family, | chr18 |
| | member 2 | |
| Pex16 | Peroxisomal biogenesis factor 16 | chr3 |
| Pgk1 | Phosphoglycerate kinase 1 | chrX |
| Polr2a | Polymerase (RNA) II (DNA directed) | chr10 |
| | polypeptide A | |
| Ppia | Peptidylprolyl isomerase A (cyclophilin A) | chr14 |
| Ppib | Peptidylprolyl isomerase B | chr8 |
| Pum 1 | Pumilio RNA-binding family member 1 | chr5 |
| Rp14 | Ribosomal protein L4 | chr8 |
| Rplp2 | Ribosomal protein, large P2 | chr1 |
| Sdha | Succinate dehydrogenase complex, subunit A, | chr1 |
| | flavoprotein (Fp) | |
| Srsf4 | Serine/arginine-rich splicing factor 4 | chr5 |
| Tbp | TATA box binding protein | chr1 |
| Tfrc | Transferrin receptor | chr11 |
| Trap1 | TNF receptor-associated protein 1 | chr10 |
| Ubc | Ubiquitin C | chr12 |
| Ywhag | Tyrosine 3-monooxygenase/tryptophan 5- | chr12 |
| | monooxygenase activation protein, gamma | |
| Ywhaz | Tyrosine 3-monooxygenase/tryptophan 5- | chr7 |
| | monooxygenase activation protein, zeta | |

In some embodiments, the intensified treatment includes at least one of: intensified radiotherapy treatment, systemic therapy or mastectomy. In some embodiments, the therapy excludes radiation therapy.

In some embodiments, treating the subject with intensified radiotherapy denotes an amount of therapy above the guidelines in the NCCN, ESMO, Clinical Practice Recommendations Australia, or NICE guideline, ignoring the PD-1 marker state. In some embodiments, the NCCN, ESMO, Clinical Practice Recommendations Australia, or NICE guideline is of 2020.

In some embodiments, treating the subject with intensified radiotherapy denotes a dose of at least one of: 67 Gy or more, add a boosting dose to a standard recommended treatment for the subject when the standard recommended treatment does not include a boosting dose, increase a boosting dose beyond the standard amount for the subject, increase the fraction dose on a per fraction basis above the standard for the subject, increase the number of fractions of a recommended dose above the standard for the subject.

In some embodiments, treating the subject comprises the standard recommended treatment from the NCCN, ESMO, Clinical Practice Recommendations Australia, or NICE guideline when the subject has elevated PD-1. In some embodiments, the guideline used is the most recent version of any of these guidelines, as of May 12, 2021.

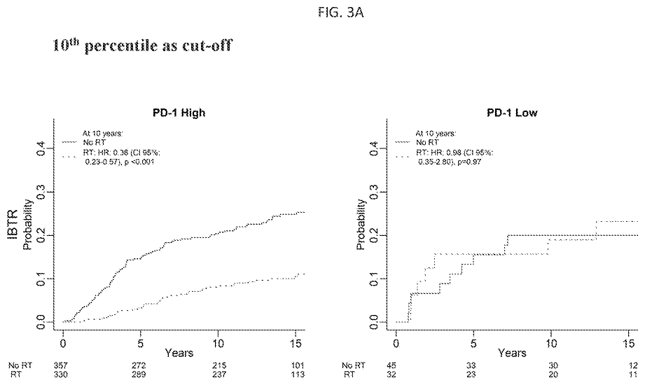

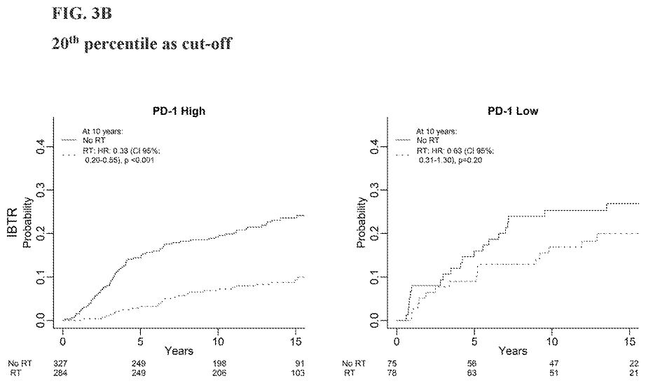

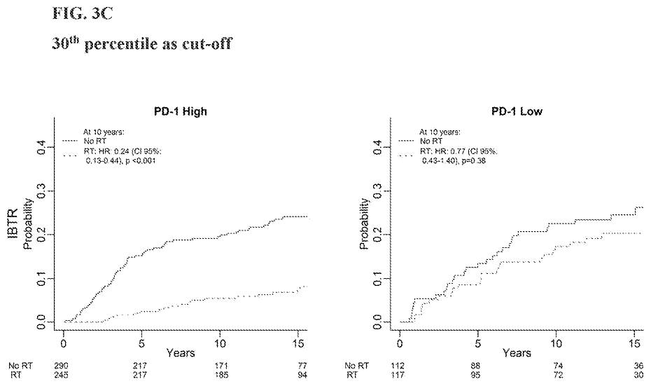

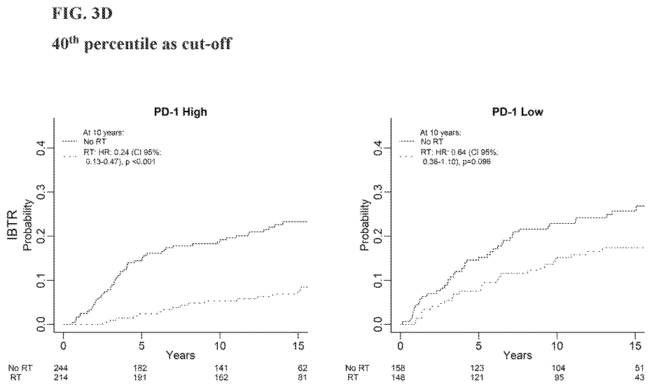

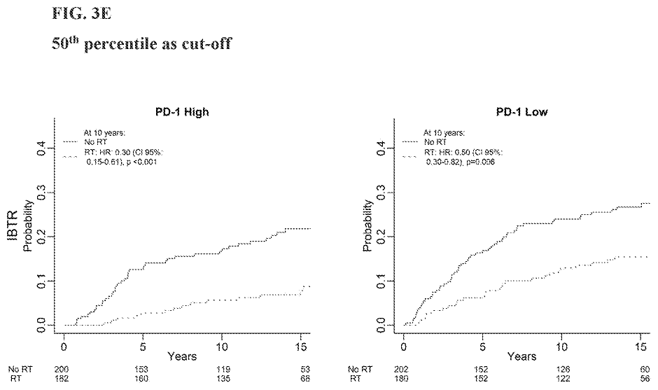

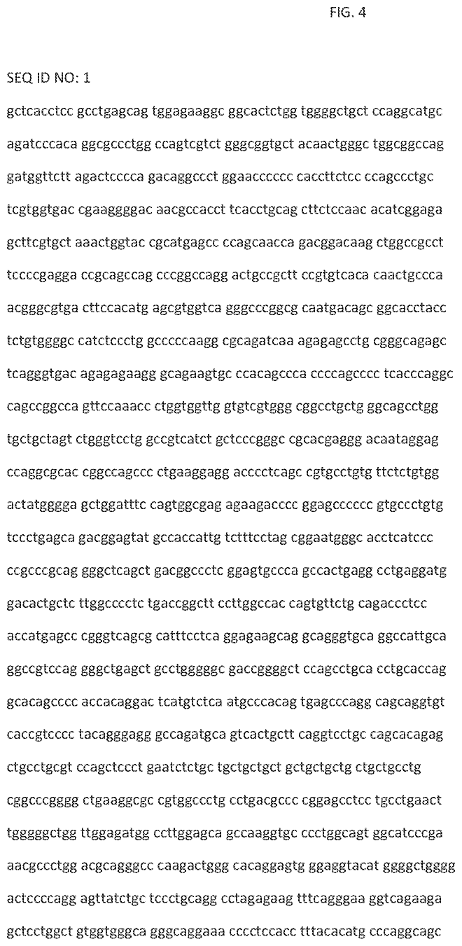

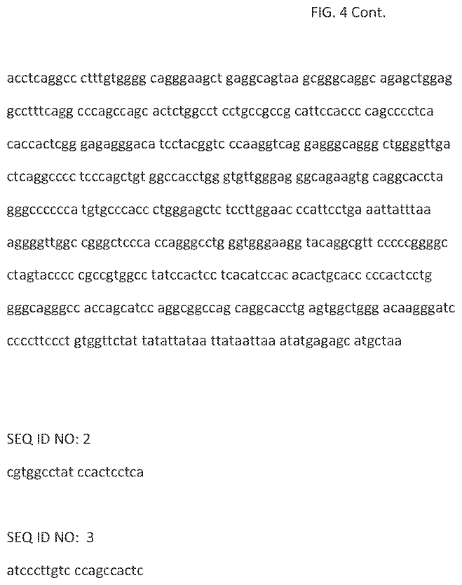

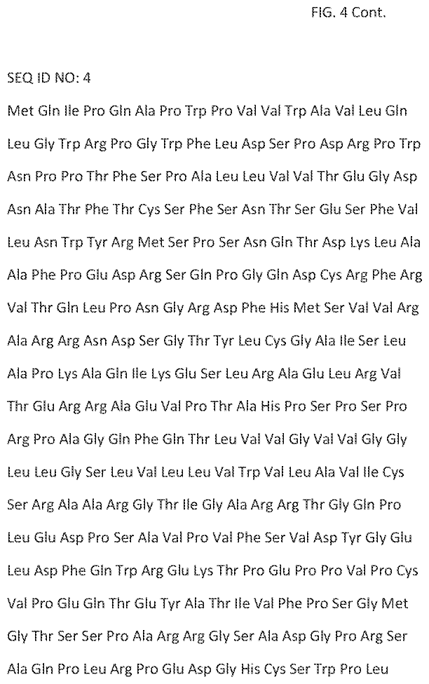

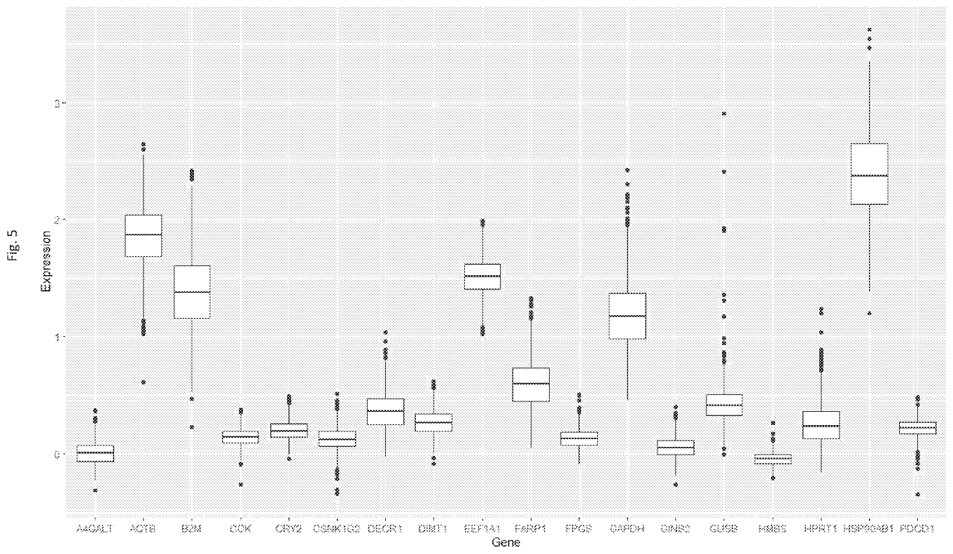

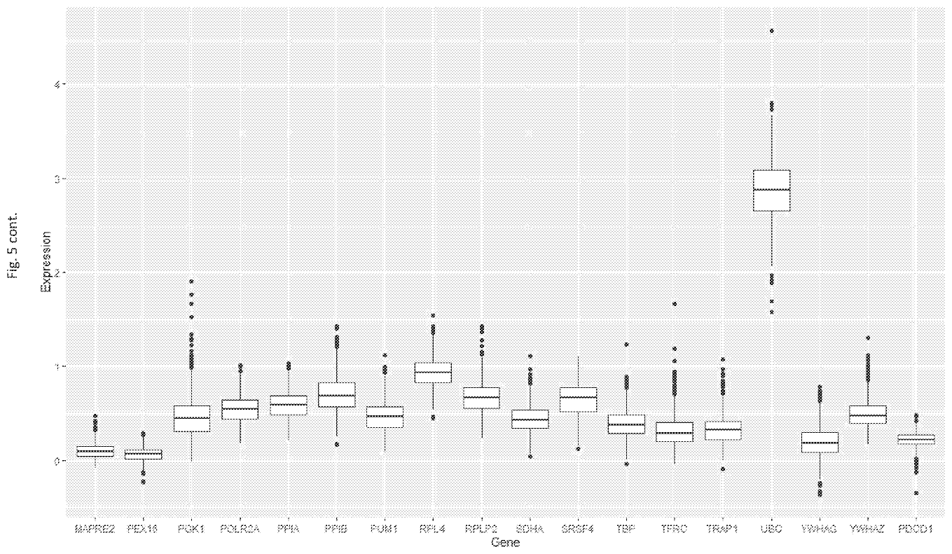

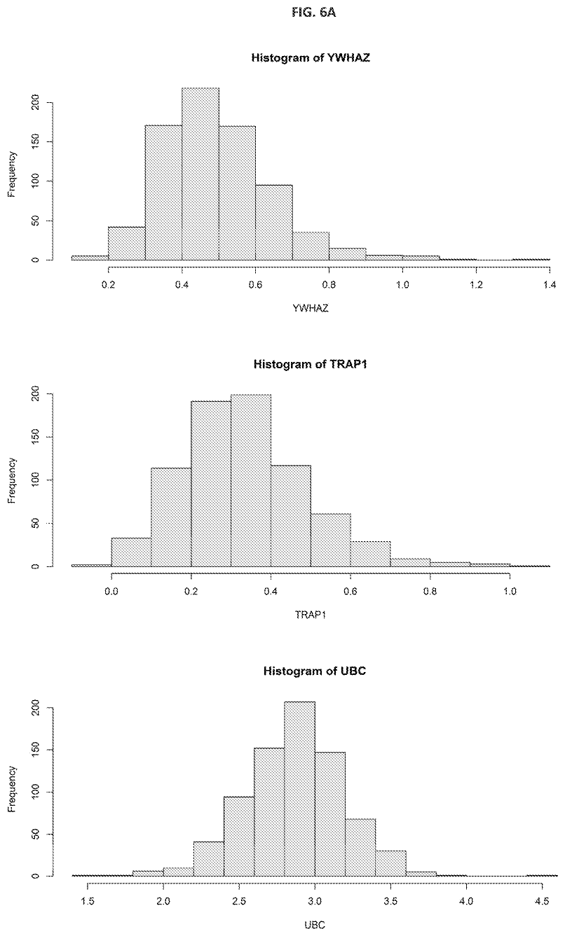

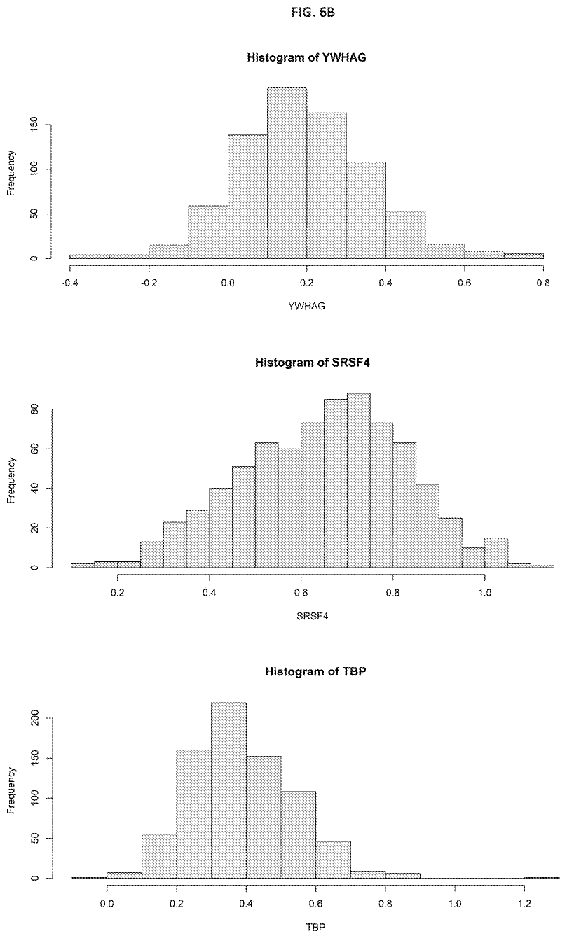

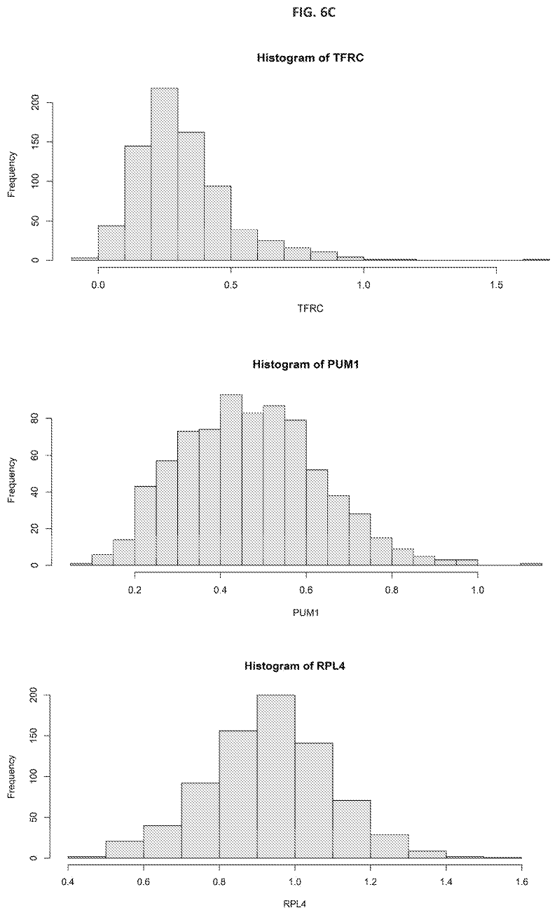

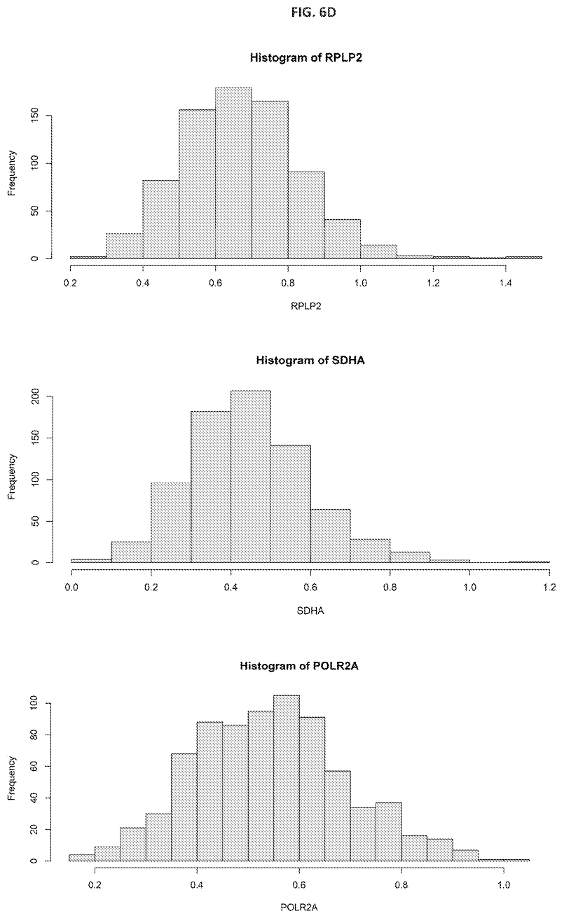

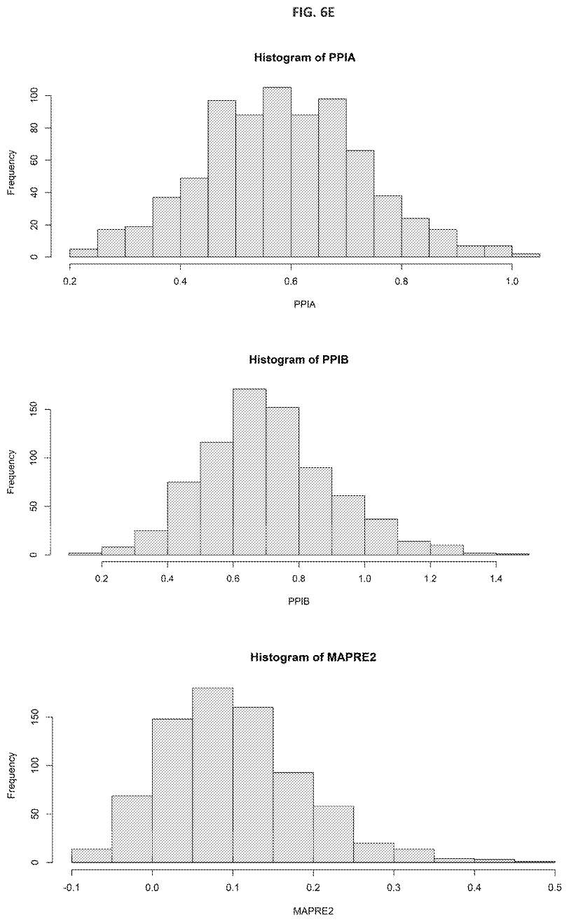

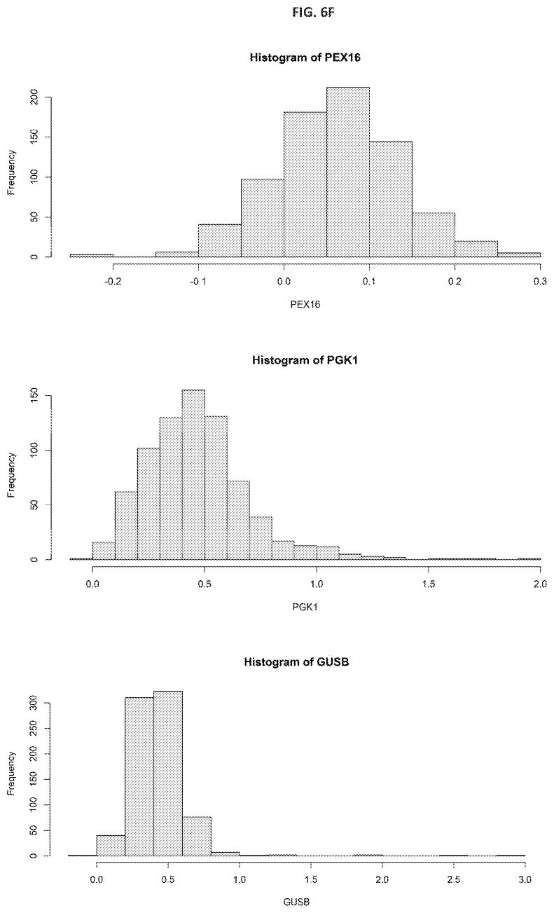

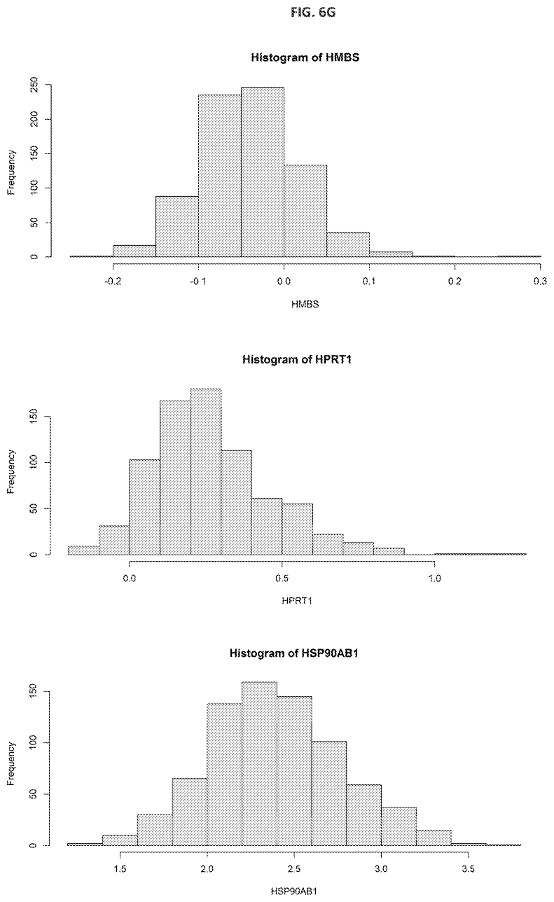

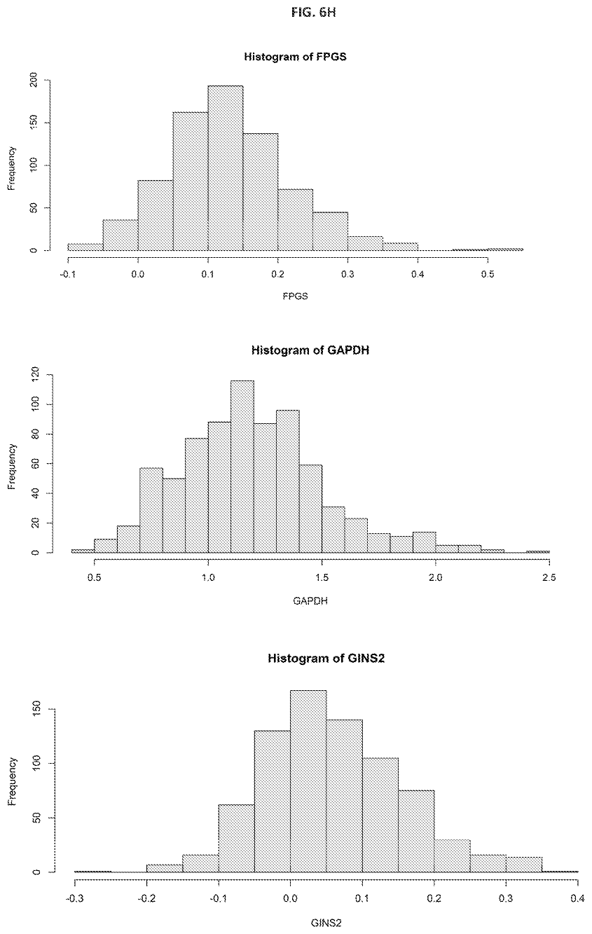

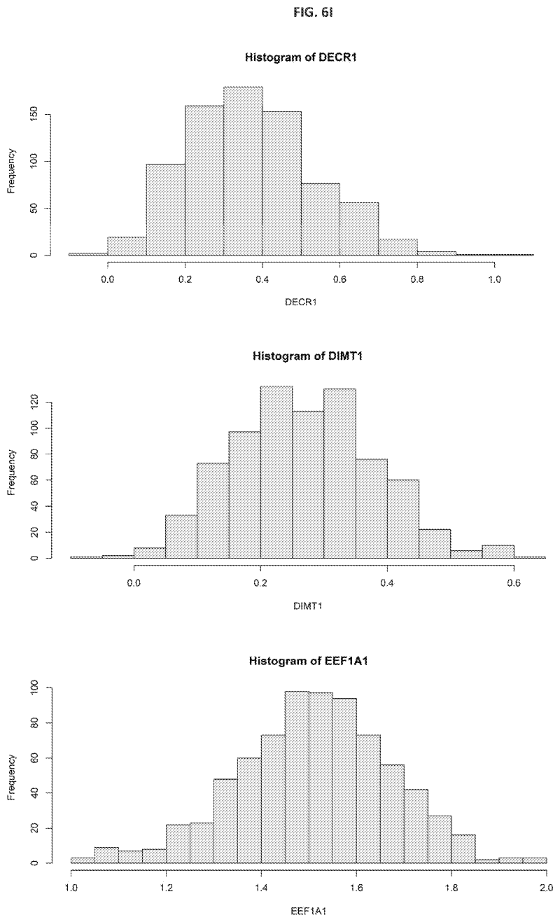

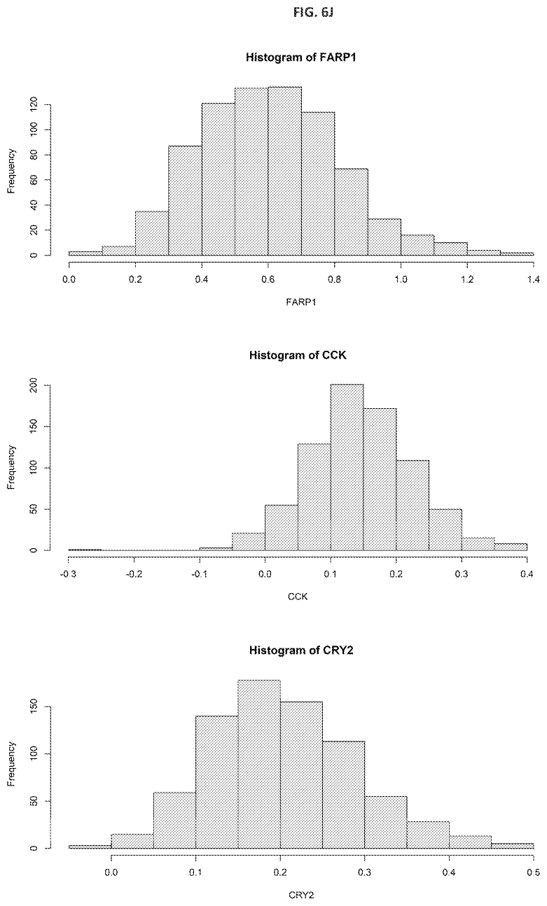

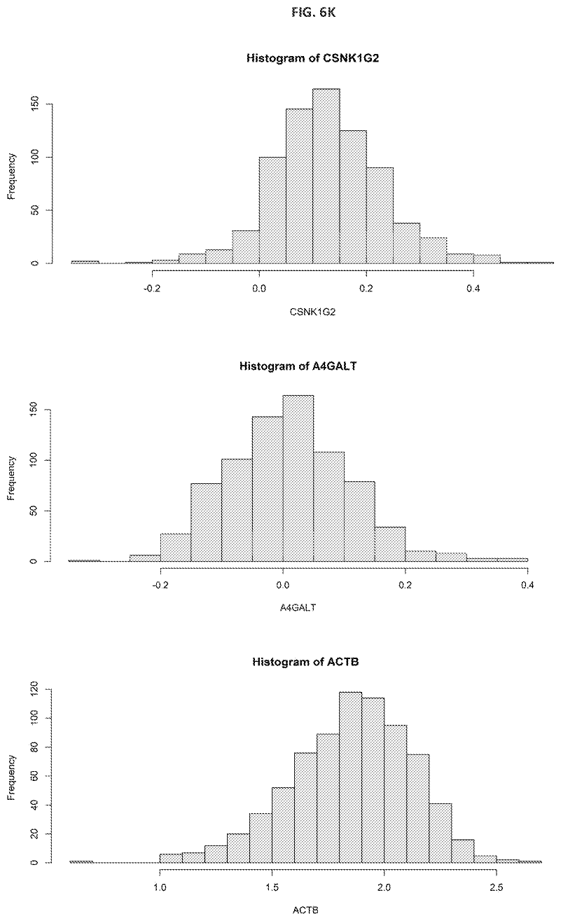

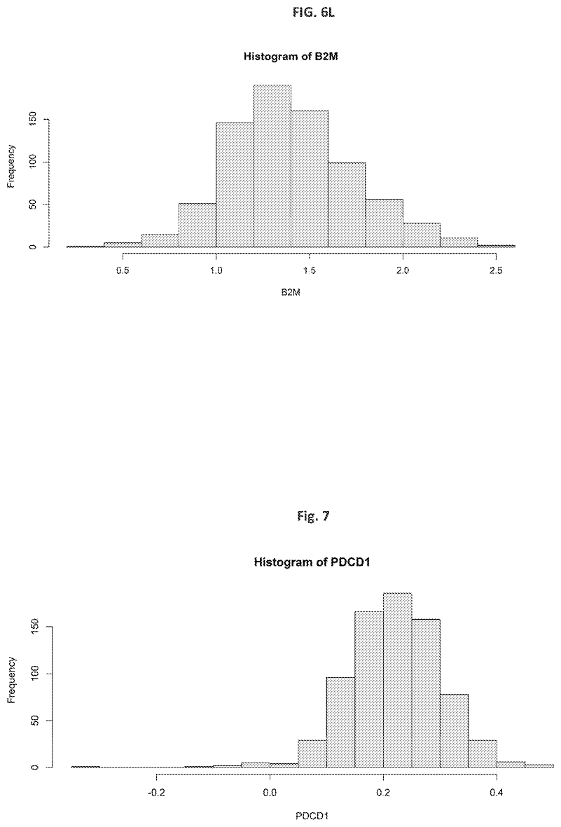

In some embodiments, low level PD-1 denotes the subject has PD-1 levels in a lowest quartile of PD-1 levels of a population of subjects having invasive breast cancer, relative to a set of one or more selected expression levels of housekeeping genes. In some embodiments, a representative “low” PD-1 level is shown in FIG. 5 (box plot, comparing various housekeeping genes to PD-1 (AKA PDCD1) and in FIG. 7 (AKA PDCD1). FIG. 6A-6L shows the histograms of various other genes of the tested population from Example 1 provided herein. In some embodiments, alternative cutoffs can be used for “low” PD-1, such as 10%, 20%, 30%, 40% and 50%—the results for such percentile cut-offs are shown in FIGS. 3A-3E.

In some embodiments, two or more housekeeping genes are used from the sample as a control comparison for PD-1 level to determine low PD-1 level.

In some embodiments, low PD-1 expression is defined as an amount less than the lowest 25% of a population of subjects having invasive breast cancer.

In some embodiments, when radiation is administered, it is administered at a biologically effective dose (BED) of 73 Gy or more with a tumor alpha/beta ratio of 5, or a BED of 78 Gy or more with a tumor alpha/beta ratio of 4, or a BED of 87 Gy or more with a tumor alpha/beta ratio of 3, or a BED of 104 Gy or more with a tumor alpha/beta ratio of 2, wherein optionally, BED can be a measure of the true biological dose delivered by a combination of dose per fraction (d) and number of fractions (n) to a tissue characterized by a specific radiosensitivity (alfa/beta ratio): BED=nd[1+d/(alfa/beta)].

In some embodiments, for patients who are not recommended a boost according to guidelines, an intensified treatment will have a maximum BED of 97 for an alpha/beta ratio of 5, 105 for an alpha/beta ratio of 4, 115 for an alpha/beta ratio of 3 and 137 for an alpha/beta ratio of 2.

In some embodiments, for patients who are recommended a boost according to the guidelines, the intensified treatment will have a maximum BED of 130 for an alpha/beta ratio of 5, 140 for an alpha/beta ratio of 4, 147 for an alpha/beta ratio of 3 and 160 for an alpha/beta ratio of 2.

In some embodiments, a level of PD-1 is analyzed as a continuous metric so that a continuous risk assessment is further provided to the subject.

In some embodiments, intensified treatment or intensified therapy denotes at least one of: intensified radiotherapy treatment, systemic therapy, mastectomy, the additional use of a sensitizer to another therapy; a therapy above a level set by a guideline, that can be, for example, a NCCN, ESMO, Clinical Practice Recommendations Australia, or NICE guideline for the subject's remaining indicators, or any combination thereof.

In some embodiments, a level of PD-1 is determined by at least one of: blot/laser capture, microdissection, RT-PCR, QPCR, PCR, deep sequencing, RNA-seq, a microarray assay, normalized and non-normalized probes, and NanoString.

In some embodiments, for any of the embodiments provided herein a) the subject is at risk of or b) the therapy or treatment is for, or c) the recurrence is, a local recurrence.

In some embodiments, for any of the embodiments provided herein a) the subject is at risk of or b) the therapy or treatment is for, or c) wherein recurrence is, a local and/or regional recurrence.

Some embodiments relate to a method of selecting a treatment for a subject; the method comprises: 1) comparing a level of PD-1 in a subject to a range of PD-1 levels; and 2) increasing a likelihood of administering radiotherapy to the subject as an inverse function of a level of PD-1, wherein a lower PD-1 level indicates a greater benefit from intensified radiotherapy to the subject, thereby decreasing a risk of local breast cancer recurrence.

In some embodiments, a sample comprises a core biopsy sample, a fine-needle aspiration (FNA) sample, an excisional biopsy sample or a sample from surgery.

In some embodiments, the method can also be used for preinvasive breast cancer (e.g ductal carcinoma in situ, DCIS). In some embodiments, the patient may be a patient that has undergone surgery, which preferably is breast-conserving surgery or mastectomy, where breast-conserving surgery is preferred. The patient is preferably a female patient in the case of breast cancer.

In some embodiments, the patient may preferably be a patient that is diagnosed with early stage breast cancer. The early stage breast cancer may be lymph node negative (NO) stage 0 (ductal cancer in situ), I or IIA cancer. The stage is determined as in known in the art of pathology. Thus, it is preferred that the tumour has a diameter less than 5 cm, and there are no macrometastases in the lymph nodes.

In some embodiments, a sample is taken from the tumour of the patient. The sample may be a biopsy which is taken before surgery or during surgery. The sample may be a breast cancer tissue biopsy.

The sample is preferably a biopsy from the operated tumor but other biopsy alternatives include core biopsy, excisional biopsy, stereotactic biopsy and fine-needle aspiration. The biopsy may comprise CD8+cells, which is the cell type that typically expresses PD-1. Hence, the biopsy may preferably comprise T-cells which have infiltrated the tumour. The sample may comprise circulating tumour cells.

In some embodiments, when the sample has been collected, it is preferably treated as is known in the art of pathology. Gene expression profiling is preferably performed on fresh frozen or formalinfixed paraffin-embedded tissue. When an antibody is used for visually detecting PD-1 the sample is preferably paraffin embedded.

In some embodiments, it is determined if the expression level is below a threshold level. Detection of PD-1 expression may be carried out using any suitable method that makes it possible to quantify expression level of PD-1.

In some embodiments, the expression level may be in relation to a reference group, where the PD-1 expression level of the members of the group has been quantified and the PD-1 expression level of the patient is compared to the group. The reference group may be a group of patients that is similar to the patient being treated (for example women with early breast cancer). The expression level in the reference group is preferably determined using the same method as for the patient. Typically, each pathology laboratory will have their own reference group.

In some embodiments the expression levels of the reference group are ranked according to PD-1 expression level and a threshold expression level is determined as the expression level of a predetermined percentile of the reference group. For example, the threshold may be determined as the expression level of the 25th percentile, where expression levels over the 25^{th} percentile are considered to be high expression. Any suitable cut-off may be used, such as the 10^{th}-40^{th}, more preferably the 10^{th}-30^{th} percentile. Hence the cut-off may be for example the 15^{th}, 20^{th}, the 25^{th} or the 30^{th} or the 40^{th} percentile. The number of patients in the reference group is preferably at least 100, more preferably at least 1000.

Hence it may be determined if the patient belongs to one of two groups, high -expressing patients or low expressing patients.

Various suitable methods may be used to detect PD-1 expression. PD-1 expression determination may be carried out at the mRNA level. PD-1 expression may for example be detected using northern blot, quantitative PCR, whole RNA sequencing, expression arrays, in situ hybridization. A useful guide to various techniques useful in the detection of macromolecules is Current Protocols in Molecular Biology, Current Protocols in Human Genetics, and current protocols in Immunology, all published by the Wiley group. Sequencing methods may include Sanger sequencing or next generation sequencing (NGS) methods. For example, total mRNA of the sample may be sequenced, for example using NGS, and the number of sequence reads is counted, where the number or sequence reads correlate with levels of PD-1 mRNA in the sample.

In some embodiments, the mRNA transcript of PD-1 is detected.

A suitable PD-1 mRNA sequence for detection may be SEQ NO 1. It is to be noted that the sequence below is described using Ts instead of Us because it is from a cDNA. The actual mRNA has uracil instead of thymidines.

The PD-1 mRNA may be extracted from the sample. Extraction can be done using any suitable method. For example, phenol extraction or using TRIzol reagent (ThermoFisher).

The method may involve using a polynucleotide probe that is able to hybridize (Watsoncrick base pair) to SEQ ID NO 1, that is, form Watson-Crick base pairs with SEQ ID NO 1. This is the case in for example array technologies, northern blot and also PCR techniques (where the probe is used to amplify the target sequence). The probe may be selected from the reverse complement sequence of SEQ ID NO 1. The polynucleotide probe is preferably specific for SEQ ID NO 1. The polynucleotide probe preferably has a length of at least 15 nucleotides, more preferably at least 18 nucleotides and even more preferred at least 20 nucleotides. The nucleotide is preferably able to hybridize to SEQ ID NO 1 in a specific manner, and preferably with high affinity under what the skilled person refers to as stringent conditions. When designing the polynucleotide probe it may be useful to BLAST it against other mRNA sequences that may be present in the sample such as human mRNA sequences and virus mRNA sequences.

Synthesis of polynucleotides are known in the art of organic chemistry. In general, a polynucleotide may be synthesized using suitable chemistry known in the art, where the individual nucleotides are added one by one. For example, a solid phase may be used. Of course, typically the polynucleotide probe is ordered from a company which specializes in oligonucleotide synthesis.

In some embodiments, PD-1 expression may be determined using quantitative PCR (qPCR), such as for example real-time PCR. One primer is preferably labelled to enable detection of the PCR products. A suitable system for quantitative PCR is the TaqMan system (Applied Biosystems/ThermoFisher). A suitable set of primers may for quantitative PCR may be the following primers:

Sense:
(SEQ ID NO 2)
cgtggcctatccactcctca.
Antisense:
(SEQ ID NO 3)
atcccttgtcccagccactc.

PD-1 expression may also be determined using northern blot where a labelled nucleotide probe is allowed to hybridize with total mRNA extracted from the sample. The amount of binding of the probe correlates with the amount of PD-1 RNA in the sample.

Alternatively, the amount of PD-1 mRNA is determined using a gene array. Total mRNA is converted to labelled cDNA and is allowed to bind to a probe that is immobilized. The amount of binding is detected using the label.

In some embodiments the polynucleotide probe, mRNA or cDNA may be labelled in order to detect binding. Suitable labelling methods include radiolabeling or fluorescence.

When it is referred to sequences ID NO herein it also comprises sequences that are at least 95%, more preferably at least 98% even more preferred at least 99% identical to the disclosed sequence. Sequence identity is calculated using BLAST2SEQUENCES, using default settings.

SEQ ID 1 represent the longer isoforms of PD-1. There are also splice variants of PD1 and the sequences of such splice variants are a subset of SEQ ID no 1.

### Treatment

The expression level of PD-1 is used to determine treatment for the cancer patient. When the expression level is below the threshold, intensified treatment is useful and is administered to the patient. Intensified radiotherapy treatment is of little value to PD-1-high expressing patients and may be avoided for such patients.

For example, if the expression level is not above the threshold, providing intensified treatment such as intensified radiotherapy treatment or systemic treatment or mastectomy for the patient.

In some embodiments, the treatment reduces the risk of cancer recurrence in particular breast cancer recurrence, in particular ipsilateral breast tumour recurrence (IBTR, also called local recurrence). Patients that express high level of PD-1 benefit from standard levels of radiation treatment, but patients that express low level of PD-1 need additional treatment. Patients that express low level of PD1 needs intensified treatment compared to patient that express high levels of PD-1. In particular the intensified treatment may comprise intensified radiation treatment.

In some embodiments, the intensified treatment may also comprise systemic therapy which may comprise chemotherapy, such as treatment with anthracyclines (e.g doxorubicin, epirubicin), taxanes (e.g paclitaxel, docetaxel), platinum-based agents (e.g carboplatin), alkylating agents (e.g cyclophosphamide), or antimetabolites (e.g 5-fluorouracil) or other chemotherapeutic agents. Treatment may also comprise antibodies used for targeting tumours, in particular breast cancer tumours. Such antibodies may include trastuzumab, pertuzumab or checkpoint blockade therapy such as Pembrolizumab or Nivolumab. In the case of breast cancer, the intensified treatment may also comprise mastectomy.

In some embodiments, breast cancer patients that express low or no levels of PD-1 are given intensified treatment. This may be provided as intensified radiotherapy treatment. In the case of radiotherapy for breast cancer, patients are treated with external breast radiotherapy or brachytherapy or intraoperative radiotherapy. External breast radiotherapy may be whole breast radiotherapy or partial breast radiotherapy, where whole breast radiotherapy is preferred.

In some embodiments (for low expressors) external radiotherapy treatment may be given as tangential opposed fields of from 4-20 MV photons more preferably 4-15 MV photons. The number of radiation fractions for external radiotherapy may be from 5 to 33 for external beam radiotherapy. It is to be noted that it is referred to “whole breast” radiotherapy after breast conserving surgery or postmastectomy radiotherapy after mastectomy has been carried out on the patient. The radiotherapy is preferably provided with a radiotherapy system which may comprise a linear accelerator that provides a radiation beam, a collimator and a treatment planning computer with a radiation dose planning tool. The radiotherapy may also be given with radiation source close to the operated tumor region(brachytherapy). The dose of radiation treatment is expressed as absorbed dose in Grays (Gy). However, the biological effect of the treatment is dependent on how the total dose is fractionated. The biological effect increases exponentially with increased dose. The biological effect is expressed as Biologically Effective Dose (BED).

In some embodiments, intensified radiotherapy treatment involves a biologically effective dose (BED) of 80 Gy or more, more preferably 85 Gy or more and most preferably 100 Gy or more, based on the formula BED=D(1+d/(α/β)) where D is the total dose in Gy, d is the dose per fraction in Gy and α/β is the characteristic constant of the tissue being referred to. Typically, α/β=4 for breast cancer. A person skilled in the field of breast radiotherapy is familiar with the concept of BED and how BED is determined for different treatment protocols.

In some embodiments, the intensified or aggressive or elevated radiotherapy treatment is one or more of whole breast external radiotherapy, partial breast radiotherapy or brachytherapy or a combination thereof, with a biologically effective dose of (BED) of 73 Gy or more with a tumor alpha/beta ratio of 5 or a BED of 78 Gy or more with a tumor alpha/beta ratio of 4 or a BED of 87 Gy or more with a tumor alpha/beta ratio of 3 or a BED of 104 Gy or more with a tumor alpha/beta ratio of 2 for patients who are not otherwise recommended a boost dose according to the current guidelines.

In some embodiments, intensified or aggressive or elevated radiotherapy treatment is one or more of whole breast external radiotherapy, partial breast radiotherapy or brachytherapy or a combination thereof, with a biologically effective dose of (BED) of 93 Gy or more with a tumor alpha/beta ratio of 5 or a BED of 100 Gy or more with a tumor alpha/beta ratio of 4 or a BED of 111 Gy or more with a tumor alpha/beta ratio of 3 or a BED of 133 Gy or more with a tumor alpha/beta ratio of 2 for patients who are recommended a boost according to the current guidelines.

In some embodiments, standard radiotherapy treatment is involves a BED of 75 Gy, or less, more preferably 70 Gy or less and most preferably 65 Gy or less, where α/β3=4.

Examples of standard radiotherapy treatments are regimens 1-4 below. This type of treatment may be useful for patients who do not have low levels of PD-1 expression.
1. 25 fractions of 2 Gy each (total 50 Gy), preferably delivered in five days per week for five weeks.
2. 15 fractions of 2.67 Gy each (total 40 Gy) preferably delivered in five days per week for three weeks.
3. 16 fractions of 2.66 Gy each (total 42.5 Gy) preferably delivered in five days per week for approximately three weeks.
4. 5 fractions of 5.2 Gy each (total 26 Gy) delivered in five days for one week.

The above treatment protocols each have a BED of <75 where a/f3=4.

Examples of intensified radiotherapy treatment may be any of treatment regimens 1-4 above in combination with one of:
5. 5-8 fractions of 2 Gy each (total 10-16 Gy) delivered in five days per week for 1-2 weeks.
6. Brachytherapy (total 10-15 Gy) delivered in 10-12 sessions.

Hence one example of an intensified radiotherapy protocol is protocol 1 in combination with protocol 5, that is, an initial treatment of 25 fractions of 2 Gy each (total 50 Gy), then a 5-8 fraction boost of 2 Gy each (total 10-16 Gy) delivered in five days per week for 1-2 weeks.

A further example of intensified radiotherapy is a simultaneously integrated boost against the operational cavity of 15 fractions of 0.53 Gy (in addition to treatment regimen 2 or 3 above resulting in 3.2 Gy fractions) (total 48 Gy) delivered in five days per week in three weeks., e.g.,first 15 fractions of 3.2 Gy, preferably given five days per week for three weeks, of which 0.53 Gy per fraction is a boost dose provided to the location where the tumour was located before it was removed by surgery.

In some embodiments, parts of the method of treatment may be implemented by using software, such as a radiation dose planning tool. With using such a tool, a user may be able to calculate a suitable radiation dose to be provided to a patient by entering suitable parameters such as PD-1 expression, age and -clinicopathological variables which predict risk of recurrence, such as histological grade, tumour size, tumour location, Ki67, estrogen/progesterone/HER2 receptor status and predictions from other prognostic or radiotherapy predictive genomic/immunohistochemistry classifiers.

There is also provided a method for diagnosis comprising the steps of a) obtaining a tissue sample of a tumour from a cancer patient, preferably a breast cancer patient, b) determining the expression level of PD-1 in the sample, c) determining that the expression level is below a threshold expression level, d) determining that the patient belongs to a group that would benefit from intensified treatment as radiotherapy treatment or systemic treatment to the patient. The method is preferably carried out outside the patient's body.

In some embodiments, the patient's age is also considered to determine whether intensified treatment will be provided to the patient. In some embodiments, the age is between 50 and 60 years old.

In some embodiments, the patient's tumor subtype is also considered to determine whether intensified treatment will be provided to the patient. In some embodiments, the patient has Luminal B tumors.

In some embodiments, a radiotherapy boost is omitted to patients with higher than threshold PD-1 expression levels and younger than 50 years old.

### Reports/Recommendations

In some embodiments, any of the present methods can further comprise preparing a report regarding the risk associated with the human invasive tissue sample. In some embodiments, the report is a written report providing the risk of invasive breast cancer. In some embodiments, the report is generated from and/or includes one or more of the marker options/combinations provided herein. In some embodiments, the report also details if the subject will be receptive to standard radiation therapy, intense radiation therapy or if a non-radiation therapy, such as an antibody to HER2, should be employed.

In some embodiments, the method further comprises recommending a treatment given a result from analyzing the sample for PD-1 levels. In some embodiments, the treatment is less aggressive than would have otherwise been recommended, without the method. In some embodiments, the treatment is more aggressive than would have otherwise been recommended, without the method. In some embodiments, the report also details if the subject will be receptive to radiation therapy or if a non-radiation therapy, such as an antibody to HER2, should be employed (e.g., depending upon the PD-1 results).

### Treatment

In some embodiments, the appropriate treatment of non-radiation or radiation therapy can be provided to the subject or received by the subject. In some embodiments, the non-radiation therapy is an antibody to HER2, such as trastuzumab. Other examples of non-radiation therapy include one or more of: immunotherapy; chemotherapy, anti-hormonal therapy, other monoclonal antibody therapies (PARP inhibitors, Cdk4/6 inhibitors etc)

In some embodiments, a therapy comprises at least one of surgical resection, radiation therapy, anti-hormone therapy. In some embodiments, a therapy can be appropriate if one knows that the subject has a low likelihood of an invasive event, but would not be appropriate if one knows that the subject has a high likelihood of an invasive breast cancer event and how likely the subject is refractory to radiation therapy.

In some embodiments, a therapy appropriate to reduce a risk of invasive breast cancer (a local recurrence of breast cancer) comprises at least one of mastectomy, targeted HERs therapy, receptor-targeted chemotherapy. In some embodiments, such a therapy can be appropriate if one knows that the subject has a high likelihood of an invasive event, but would not be appropriate if one knows that the subject has a low likelihood of an invasive breast cancer event. In some embodiments, the therapy is appropriate if the subject is not, non-responsive to the therapy. In some embodiments, a subject who is predicted to be refractory to radiation therapy will not receive or be administered a radiation therapy (or will receive an elevated level of radiation therapy to make up for their poor response to the radiation therapy).

In some embodiments, any of the above methods when applied to DCIS can be followed by “watchful waiting” or other relatively minimal/intrusive therapies.

In some embodiments, any of the methods provided herein can be applied to DCIS and/or invasive breast cancer for the successfulness of the therapy in preventing a recurrence of the event (e.g., either invasive breast cancer or DCIS).

Additional aspects and approaches regarding possible therapeutic actions that are specific for the present invasive breast cancer subjects are provided below.

In some embodiments, a kit is provided. The kit can include a PD-1 probe, and, optionally, one or more other probes. In some embodiments, the probe is an isolated antibody. In some embodiments, the probe is a nucleic acid that selectively hybridizes to PD-1 as appropriate. In some embodiments, the kit contains enough of the probe and/or the probe is sensitive and/or selective enough such that the “+” and “−” states of PD-1.

In some embodiments, a solid support comprising probes specific for at least PD-1 is provided. In some embodiments, the probes consist essentially of probes or antibodies specific for the prediction of responsiveness to radiotherapy. In some embodiments, a solid support comprising probes specific for at least PD-1 is provided.

In some embodiments, the subject and/or sample to be analyzed can be a patient (or from a patient).

In some embodiments, the invasive breast cancer sample itself can be processed in any number of ways to prepare it for screening for the markers. In some embodiments, the invasive breast cancer sample has been surgically removed from a patient and preserved. In some embodiments, the sample is obtained by surgical removal. In some embodiments, the sample is cut into one or more blocks, such as 2, 3, 4, 5 or more blocks.

In some embodiments, a signature comprising a level of PD-1 is at least one of: an RNA level, a DNA level, or some combination thereof.

In some embodiments, a method of preparing a sample is provided. The method comprises obtaining a sample from a subject and preparing it so that its DNA, RNA, can be analyzed for at least PD-1.

In some embodiments, the sample is preserved. In some embodiments, the sample is preserved via freezing. In some embodiments, the sample goes through (or does not go through) embedding in a chemical such as Optimal Cutting Temperature (OCT) compound, or fixation with a chemical(s), including, without limitation, formalin, formaldehyde, quaternary ammonium salts, alcohol, acetone, or other chemicals that preserve or extract DNA or RNA. In some embodiments, the technique used is one that allows PD-1 DNA or RNA to be preserved in an adequate amount and state so that PD-1 can be analyzed as provided herein.

In some embodiments, analyzing the sample comprises determining an amount of a specified RNA in the sample. The amount of RNA for each marker can be determined by any number of techniques, some of which are discussed elsewhere in the present application. In some embodiments, the RNA level is determined by at least one of: an assay involving nucleic acid microarray, reverse transcriptase-polymerase chain reaction, in situ nucleic acid detection, or a next generation sequencing method. In some embodiments, expression of at least PD-1 is measured by real time quantitative polymerase chain reaction or microarray analysis.

In some embodiments, the RNA level is determined by: an assay involving nucleic acid microarray, reverse transcriptase-polymerase chain reaction, in situ nucleic acid detection, and/or a next generation sequencing method.

In some embodiments, a sample can be prepared by a certified breast pathologist confirming cancer content in the samples. A representative tumor area can then be outlined on a H&E (hematoxylin and eosin) stained slide. The RNeasy FFPE kit (Qiagen, Hilden, Germany) RNA was used to extract RNA from 1.5 mm tissue punches (in the present examples) . The Ovation FFPE WTA system (NuGEN, San Carlos, CA) was used to amplify cDNA. The Encore Biotin Module (NuGEN, San Carlos, CA) was used to fragment and label amplified cDNA which was then hybridized to GeneChip Human Exon 1.0 ST Arrays (Thermo Fisher Scientific, South San Francisco, CA). Gene expression was normalized using Single Channel Array Normalization (Piccolo SR, Sun Y, Campbell JD, et al: A single-sample microarray normalization method to facilitate personalized-medicine workflows. Genomics 100:337-44, 2012.). Sample processing can be performed in a CLIA-certified clinical operations laboratory (GenomeDx Inc, San Diego, CA). In some embodiments, different gene expression methods can be employed, including: blot/laser capture, microdissection, RT-PCR, QPCR, PCR, deep sequencing, RNA-seq, a microarray assay, normalized and non-normalized probes, and NanoString. In some embodiments, the gene expression data can then either be normalized and compared to one or many reference (housekeeping) genes or compared to one or many reference populations consisting of breast cancer patients with early breast cancer.

In some embodiments, patient specimens used for the detection of the biomarkers can be surgically removed breast tissues that are cut into small blocks and submerged in fixative. In some embodiments, following fixation, the blocks can be dehydrated and then embedded in paraffin wax. In some embodiments, the small blocks are no more than 20 mm in length and 5 mm in thickness to allow complete penetration of the fixative. In some embodiments, the fixation occurs in 10% neutral-buffered formalin for 24 to 48 hours at room temperature to preserve tissue structure and compartmentalization of the various markers. However, other fixatives and fixation times (e.g., 6 to 72 hours) can also be compatible with the marker assays. In some embodiments, assays are optimized to use specimens that have been flash frozen (e.g., in liquid nitrogen), rather than being fixed and embedded.

In some embodiments, the process of sample processing can include dehydration and embedding, which can be done manually or automated with a tissue processing instrument. In either case, the aqueous portion of the tissue and the fixation solution can be replaced by passing the block through a series of increasingly concentrated alcohol solutions. After reaching 100% alcohol, the alcohol is replaced using a chemical like xylene (or a xylene-free equivalent), followed by introduction of molten, low-melting-temperature (e.g., approximately 45° C.) paraffin wax for embedding. The FFPE blocks can be stored for many years prior to analysis. In some embodiments, “cores” of DCIS tissue can be cut from these blocks using a hollow needle and then inserted in an array format in a separate block of paraffin. Such “tissue microarrays” (TMAs) allow assessment of multiple tissues on a single section/microscope slide.

In some embodiments, ultrathin sections, approximately three to five micrometers in thickness, can be cut off the formalin-fixed paraffin-embedded (FFPE) tumor blocks using a microtome. The sections can be mounted onto glass microscope slides, ensuring that the tissue does not become folded or fragmented, which could interfere with the assays. The glass microscope slides can contain a positively charged surface in order bind to the negatively charged tissue sections, although other methods of tissue binding, including adhesives, can also be compatible.

In some embodiments, wax removal and rehydration of the tissue sections can then be carried out. These processes can be done manually or automated with certain staining instruments. Wax can be removed from the tissue sections on the slides through heating and/or immersion in a solution of xylene (or an equivalent xylene-free solution, such as Novocastra Bond Dewaxing Solution). Rehydration can be accomplished by passing the slides through a series of decreasingly concentrated alcohol solutions until a concentration of 0% is reached (pure water). Following wax removal and rehydration, the tissue sections can be stained with hematoxylin and eosin (H&E) and for a variety of molecular markers using immunohistochemistry (IHC) and/or in situ hybridization (ISH) assays and then assessed by pathologists or histotechnologists, as described below. The above processing steps can be performed for any of the methods provided herein in regard to the PD-1 marker.

Any one or more of the following arrangements is also contemplated:
1a. A method for treating breast cancer comprising the steps:
a) obtaining a tissue sample of a tumour from a breast cancer patient,
b) determining the expression level of PD-1 in the sample,
c) determining that the expression level is below a threshold level,
d) providing intensified treatment as radiotherapy treatment, systemic therapy or mastectomy to the patient.
2a. The method of arrangement la where the intensified treatment comprises radiotherapy treatment.
3a. The method of arrangement la where the intensified treatment comprises systemic therapy.
4a. The method of arrangement 2a where the radiotherapy treatment is whole breast external radiotherapy or brachytherapy or a combination thereof, with a biologically effective dose of (BED) of 80 Gy or more.
5a. The method of arrangement la where the patient has been subjected to breast conserving surgery or total mastectomy.
6a. The method of arrangement la where the breast cancer is an early stage breast cancer.
7a. The method of arrangement la where the expression level of PD-1 is determined by detecting the amount of PD-1 mRNA in the sample.
8a. The method of arrangement 7a where the PD-1 mRNA comprises the nucleotide sequence of SEQ ID NO 1.
9a. A PD-1 mRNA-binding nucleotide or a PD-1 antibody for use in the diagnosis of breast cancer, where the nucleotide or the antibody is used for quantifying the level of PD-1 that is expressed in a breast cancer sample, and where low expression of PD-1 indicates that the patients belongs to a patient subgroup where intensified radiotherapy treatment is needed.
10a. A method of diagnosis comprising the steps of
a) obtaining a tissue sample of a tumour from a breast cancer patient,
b) determining the expression level of PD-1 in the sample,
c) determining that the expression level is below a threshold expression level,
d) determining that the patient belongs to a group that would benefit from intensified radiotherapy treatment.
1. A method for treating breast cancer (both invasive and in situ) comprising the steps:
a) obtaining a tissue sample of a tumour from a breast cancer patient,
b) determining the expression levels of PD-1 mRNA in the sample,
c) determining that the expression level is below a threshold level,
d) providing intensified treatment as intensified radiotherapy treatment, intensified systemic therapy or mastectomy to the patient.
2. The method of arrangement 1 where the intensified treatment comprises intensified radiotherapy treatment.
3. The method of arrangement 1 where the intensified treatment comprises systemic therapy.
4. The method of arrangement 2 where
a) for a subject with no boost otherwise recommended, the intensified radiotherapy treatment is whole breast external radiotherapy, partial breast radiotherapy or brachytherapy or a combination thereof, with a biologically effective dose of (BED) of 73 Gy or more with a tumor alpha/beta ratio of 5 or a BED of 78 Gy or more with a tumor alpha/beta ratio of 4 or a BED of 87 Gy or more with a tumor alpha/beta ratio of 3 or a BED of 104 Gy or more with a tumor alpha/beta ratio of 2; or
b) for a subject with a boost otherwise recommended, the intensified radiotherapy treatment is one or more of whole breast external radiotherapy, partial breast radiotherapy or brachytherapy or a combination thereof, with a biologically effective dose of (BED) of 93 Gy or more with a tumor alpha/beta ratio of 5 or a BED of 100 Gy or more with a tumor alpha/beta ratio of 4 or a BED of 111 Gy or more with a tumor alpha/beta ratio of 3 or a BED of 133 Gy or more with a tumor alpha/beta ratio of 2 for patients who are recommended a boost according to the current guidelines.
5. The method of any one of the preceding arrangements, where the patient has been subjected to breast conserving surgery or total mastectomy.
6. The method of any one of the preceding arrangements, where the breast cancer is an early stage invasive breast cancer or breast cancer in situ.
7. The method of any one of the preceding arrangements, where the expression level of PD-1 is determined by detecting the amount of PD-1 mRNA in the sample.
8. The method of arrangement 7 where the PD-1 mRNA comprises the nucleotide sequence of SEQ ID NO 1.
9. The method of any one of the preceding arrangements, where BED is as a measure of the true biological dose delivered by a combination of dose per fraction (d) and number of fractions (n) to a tissue characterized by a specific radiosensitivity (alfa/beta ratio): BED=nd[1+d/(alfa/beta)].
10. A PD-1 mRNA-binding nucleotide for use in the diagnosis of breast cancer, where the nucleotide is used for quantifying the level of PD-1 that is expressed in a breast cancer sample, and where low expression of PD-1 indicates that the patients belong to a patient subgroup where intensified radiotherapy treatment is needed.
11. A method of diagnosis comprising the steps of
a) obtaining a tissue sample of a tumour from a breast cancer patient,
b) determining the expression level of PD-1 mRNA in the sample,
c) determining that the expression level is below a threshold expression level,
d) thereby determining that the patient belongs to a group that would benefit from intensified radiotherapy treatment; and
e) optionally providing the intensified radiotherapy treatment to the patient.
   12. A method of treating a subject, the method comprising:
   identifying an incremental risk to a subject with invasive breast cancer or in situ breast cancer of a local or regional recurrence of an invasive breast cancer based on a level of PD-1 in a sample of an invasive breast cancer in the subject; and
   administering an intensified breast cancer therapy to the subject based upon the incremental risk, wherein a higher incremental risk will increase:
a) a likelihood of an aggressive breast cancer therapy that is at least more than what would be recommended by the NCCN;
b) the aggressiveness of the aggressive breast cancer; or
c) both a) and b).
   13. A method for treating a subject for recurrence of invasive breast cancer, said method comprising:
   providing a cancer tissue sample from a subject who has invasive breast cancer;
   analyzing the cancer tissue sample for a level of PD-1; and
   treating the subject with an intensified treatment if the cancer tissue sample has a low level of PD-1.
   14. A method of treating a subject, the method comprising:
   identifying a subject with invasive breast cancer that has a low level of PD-1; and
   administering an intensified treatment to the invasive breast cancer.
   15. A method for recommending a treatment to a subject, said method comprising:
   analyzing a cancer tissue sample for a level of PD-1 from a subject; and
   recommending that one treat the subject with an intensified treatment if the cancer tissue sample has a low level of PD-1.
   16. A method for preventing an invasive breast cancer recurrence in a subject, the method comprising:
   providing a cancer tissue sample from a subject who has invasive breast cancer;
   analyzing the cancer tissue sample for a level of PD-1; and
   administering an intensified treatment if the cancer tissue sample has a low level of PD-1.
   17. A method for preventing an invasive breast cancer recurrence in a subject, the method comprising:
   receiving an intensified treatment if a cancer has a low level of PD-1.
   18. A method of modifying a treatment for a subject, the method comprising:
   identifying a subject with invasive breast cancer that has a low level of PD-1; and
   administering a breast cancer therapy to the subject, wherein the breast cancer therapy is more aggressive than a traditional breast cancer therapy, wherein the traditional breast cancer therapy is one recommended for the subject, based on the subject's risk factors excluding PD-1 levels.
   19. The method of any one of the preceding arrangements, wherein low level of PD-1 denotes a level of mRNA present in the sample.
   20. The method of any one of the preceding arrangements, wherein low level of PD-1 is defined by a comparison of PD-1 levels from the tissue sample to a control sample that does not include invasive cancer.
   21. The method of any one of the preceding arrangements, wherein low PD-1 is determined by comparison to an internal control in the sample.
   22. The method of arrangement 21, wherein the control includes a cell population that has no PD-1 expression.
   23. The method of any one of the preceding arrangements, comprising an external control that is a cell line known to stain negative for PD-1.
   24. The method of any one of the preceding arrangements, comprising an external control known to stain positive for PD-1 due to a high level of PD-1.
   25. The method of any one of the preceding arrangements, wherein a low levels of PD-1 is defined by a comparison to a standardized level set by a level of expression of a set of one or more housekeeping gene.
   26. The method of any one of the preceding arrangements, wherein the intensified treatment includes at least one of: intensified radiotherapy treatment, systemic therapy or mastectomy.
   27. The method of any one of the preceding arrangements, wherein treating the subject with intensified radiotherapy denotes a therapy above the guidelines in the NCCN, ESMO, Clinical Practice Recommendations Australia, or NICE guideline, ignoring the PD-1 marker state.
   28. The method of arrangement 27, wherein the NCCN, ESMO, Clinical Practice Recommendations Australia, or NICE guideline is of 2020.
   29. The method of any one of the preceding arrangements, wherein treating the subject with intensified radiotherapy denotes a dose of at least one of: 67 Gy or more, add a boosting dose to a standard recommended treatment for the subject when the standard recommended treatment does not include a boosting dose, increase a boosting dose beyond the standard amount for the subject, increase the fraction dose on a per fraction basis above the standard for the subject, increase the number of fractions of a recommended dose above the standard for the subject.
   30. The method of any one of the preceding arrangements, wherein low levels of PD-1 denotes the subject has PD-1 levels in a lowest quartile of PD-1 levels of a population of subjects having invasive breast cancer, relative to a set of one or more selected expression levels of housekeeping genes.
   31. The method of any one of the preceding arrangements, wherein two or more housekeeping genes are used from the sample as a control comparison for PD-1 level to determine low PD-1 level.
   32. The method of any one of the preceding arrangements, wherein low PD-1 expression is defined as an amount less than the lowest 25% of a population of subjects having invasive breast cancer.
   33. The method of any one of the preceding arrangements, wherein, when radiation is administered, it is administered at
   a) (for a subject with no boost otherwise recommended) a biologically effective dose of (BED) of 73 Gy or more with a tumor alpha/beta ratio of 5, or
      a BED of 78 Gy or more with a tumor alpha/beta ratio of 4, or
      a BED of 87 Gy or more with a tumor alpha/beta ratio of 3, or
      a BED of 104 Gy or more with a tumor alpha/beta ratio of 2; or
   b) (for a subject with a boost otherwise recommended) a biologically effective dose of (BED) of 93 Gy or more with a tumor alpha/beta ratio of 5 or a BED of 100 Gy or more with a tumor alpha/beta ratio of 4 or a BED of 111 Gy or more with a tumor alpha/beta ratio of 3 or a BED of 133 Gy or more with a tumor alpha/beta ratio of 2 for patients who are recommended a boost according to the current guidelines,
      wherein optionally, BED can be a measure of the true biological dose delivered by a combination of dose per fraction (d) and number of fractions (n) to a tissue characterized by a specific radiosensitivity (alfa/beta ratio): BED=nd[1+d/(alfa/beta)].
   34. The method of any one of the preceding arrangements, wherein a level of PD-1 is analyzed as a continuous metric so that a continuous risk assessment is further provided to the subject.
   35. The method of any one of the preceding arrangements, wherein intensified treatment or intensified therapy denotes at least one of: intensified radiotherapy treatment, systemic therapy, mastectomy, the additional use of a sensitizer to another therapy; a therapy above a level set by a guideline, that can be, for example, a NCCN, ESMO, Clinical Practice Recommendations Australia, or NICE guideline for the subject's remaining indicators, or any combination thereof.
   36. The method of any one of the preceding arrangements, wherein a level of PD-1 is determined by at least one of: laser capture, microdissection, RT-PCR, QPCR, PCR, deep sequencing, RNA-seq, a microarray assay, normalized and non-normalized probes, and NanoString.
   37. The method of any one of the preceding arrangements, wherein a) the subject is at risk of or b) the therapy or treatment is for, or c) wherein recurrence is, a local recurrence.
   38. The method of any one of the preceding arrangements, wherein a) the subject is at risk of or b) the therapy or treatment is for, or c) wherein recurrence is, a local and/or regional recurrence.
   39. A method of selecting a treatment for a subject, the method comprising:
   comparing a level of PD-1 in a subject to a range of PD-1 levels; and
   increasing a likelihood of administering radiotherapy to the subject as an inverse function of a level of PD-1, wherein a lower PD-1 level indicates a greater benefit of radiotherapy to the subject, thereby decreasing a risk of local breast cancer recurrence.
   40. The method of any one of the preceding arrangements, wherein a sample comprises a core biopsy sample, a fine-needle aspiration (FNA) sample, an excisional biopsy sample or a sample from surgery.
   41. The method of any of the preceding arrangements, wherein PD-1 expression is low if PD-1 mRNA levels below the 25th percentile of a breast cancer reference population that is GEO accession number GSE119295.
   42. The method of any of the preceding arrangements, wherein at least one of a-i below is applied:
a) In the context of local recurrence,
b) Testing on luminal tumors,
c) For the use of predicting, via PD-1 mRNA, the effectiveness of radiotherapy,
d) Not employing an analysis of PD-1 for it prognostic effect,
e) Examining early breast tumors with no lymph node metastases and which are less than 5 cm in size,
f) Not using a preexisting solid tumor (which mimics preoperative radiotherapy),
g) Using the predictive effect (i.e., without the use of checkpoint inhibitors) of PD-1 mRNA for postoperative radiotherapy,
h) Analyzing a sample that is from a postoperative subject,
i) Any combination of the above.

Any of the arrangements above applied for determining the effectiveness of radiotherapy for local cancer recurrence.

The above aspects can be applied to any of the embodiments, arrangements, or claimed inventions provided herein.

In some embodiments, the embodiments provided herein can provide some advantage or distinction over other arrangements or technologies. For example, in head and neck squamous cell carcinoma (HNSCC), human papilloma virus (HPV) positivity has long been recognized as a marker of and improved prognosis and radiosensitivity[1]. HPV is a virus and therefore, unsurprisingly, HPV positivity is associated with immune infiltration and the presence of immune-related biomarkers among HNSCC patients. Previous studies have shown that PD-1 expression is higher in HPV positive HNSCC compared to HPV negative HNSCC[2]. An association between PD-1 expression or immune cell infiltration and radiosensitivity in HNSCC[3, 4] is therefore best explained by an association with HPV positivity. This is further supported by a study by Fiedler et al which showed that the benefit from radiotherapy was strongly related to HPV positivity (defined as p16 positivity) and not PD-1 positivity[5]. The authors state the following in the discussion: “In the present study, we could not find a direct association of PD-1 with irradiation response or survival”[5]. Li et al found that PD-1 expression was associated with radioresistance in HNSCC[6] which further supports the conclusion that PD-1 expression is not an independent marker of radiosensitivity. The present findings are based on breast cancer patients and breast cancer is not associated with HPV infection. Therefore, previous findings regarding HNSCC and PD-1 expression being associated with a reduced risk of recurrence, most likely due to the association between PD-1 expression and HPV positivity, cannot be translated to breast cancer. Furthermore, studies on HNSCC often refer to radiotherapy of a preexisting tumor which is distinct from postoperative radiotherapy further contrasting the present findings to previous findings on HNSCC.

Previous studies have investigated the combination of immune checkpoint inhibitors (e.g. monoclonal antibodies targeting the PD-1 protein, thereby blocking PD-1 signaling). These studies show that concurrent inhibition of PD-1 with radiotherapy increases the benefit from radiotherapy[7]. These findings contrast the present findings which instead show that that an active PD-1 pathway (measured as an increase in PD-1 mRNA) predicts an increased benefit from radiotherapy independent of PD-1 inhibition. Studies similar to those of Manukian et al[7] show that decreasing the activity of the PD-1 pathway, through administration of monoclonal antibodies blocking the PD-1 receptor, is associated with an improved benefit from radiotherapy. In direct contrast to this, we found that high baseline PD-1 activity was predictive of an increased benefit from radiotherapy and that low baseline activity predicted a reduced benefit.

Furthermore, all animal model studies, and in vitro studies refer to irradiation of a preexisting solid tumor. This is comparable to preoperative radiotherapy. Contrary to this, the present findings relate to postoperative radiotherapy. Preoperative and postoperative radiotherapy have distinctly different effects on the tumor biology which seriously limits the utility of extrapolations from studies investigating preoperative radiotherapy to the postoperative setting.

In breast cancer, PD-1 mRNA expression has been associated with an improved overall- and disease-free survival in the triple negative subtype[8, 9]. The present study focused on local recurrences unlike previous studies investigating PD-1 mRNA as a biomarker which contrasts the present findings to those made by others. Furthermore, the present study did not focus on triple-negative tumors. Instead, luminal tumors (characterized by estrogen receptor expression) were the dominant tumor subtype, representing the vast majority of included tumor types. The association between immune-related biomarkers and prognosis have been shown to differ in non-luminal (estrogen receptor negative) [8, 9] compared to luminal tumors[10, 11]. Thus, results from studies on triple-negative tumors (which belong to the non-luminal category) cannot be extrapolated to luminal tumors. The present findings of PD-1 mRNA being associated with an improved benefit from radiotherapy should also be contrasted to previous findings regarding immune related biomarkers which have instead been associated with an unfavorable outcome among luminal tumors[10, 11]. Finally, the present study focused on the predictive value of PD-1 mRNA in contrast to its prognostic effect. As far as the inventors are aware, no art exists where PD-1 mRNA has been studied as a biomarker predictive of radiotherapy benefit in breast cancer with local recurrences as outcome.

Other factors which contrast the present findings from the state of the art is the focus on early breast tumors with no lymph node metastases and which are less than 5 cm in size. The present exercise studied the predictive effect of PD-1 mRNA for radiotherapy benefit while the all clinical studies regarding PD-1 mRNA in breast cancer have investigated the prognostic effect. Finally, all preclinical cancer studies regarding PD-1 mRNA and its interaction with radiotherapy (when combined with immune checkpoint blockade) have used radiotherapy of a preexisting solid tumor (which mimics preoperative radiotherapy) while the present study investigated the pure predictive effect (i.e., without the use of checkpoint inhibitors) of PD-1 mRNA for postoperative radiotherapy.

In some embodiments, any of the PD-1 related methods provided herein can be applied in one or more of the following contexts, based on the above aspects:
a) Focused on local recurrence,
b) Testing of luminal tumors,
c) Focused for the use of predicting, via PD-1 mRNA, the effectiveness of radiotherapy,
d) Not employing an analysis of PD-1 for it prognostic effect
e) Examining early breast tumors with no lymph node metastases and which are less than 5 cm in size,
f) Not using a preexisting solid tumor (which mimics preoperative radiotherapy),
g) Using the predictive effect (i.e., without the use of checkpoint inhibitors) of PD-1 mRNA for postoperative radiotherapy,
h) Analyzing a sample that is from a postoperative subject,
i) Any combination of the above,

The above aspects can be applied to any of the embodiments, arrangements, or claimed inventions provided herein.
1. Liu, C., et al., The molecular mechanisms of increased radiosensitivity of HPV-positive oropharyngeal squamous cell carcinoma (OPSCC): an extensive review. J Otolaryngol Head Neck Surg, 2018. 47(1): p. 59.
2. Chen, S. W., et al., Expression of PD-1/PD-L1 in head and neck squamous cell carcinoma and its clinical significance. Int J Biol Markers, 2019. 34(4): p. 398-405.
3. Lyu, X., et al., PD-1 and PD-L1 Expression Predicts Radiosensitivity and Clinical Outcomes in Head and Neck Cancer and is Associated with HPV Infection. J Cancer, 2019. 10(4): p. 937-948.
4. Fiedler, M., et al., Infiltrating immune cells are associated with radiosensitivity and favorable survival in head and neck cancer treated with definitive radiotherapy. Oral Surg Oral Med Oral Pathol Oral Radiol, 2020. 129(6): p. 612-620.
5. Fiedler, M., et al., Biological predictors of radiosensitivity in head and neck squamous cell carcinoma. Clin Oral Investig, 2018. 22(1): p. 189-200.
6. Li, G., et al., Comprehensive analysis of radiosensitivity in head and neck squamous cell carcinoma. Radiother Oncol, 2021. 159: p. 126-135.
7. Manukian, G., et al., Combining Radiation and Immune Checkpoint Blockade in the Treatment of Head and Neck Squamous Cell Carcinoma. Front Oncol, 2019. 9: p. 122.
8. Lu, L., Y. Bai, and Z. Wang, Elevated T cell activation score is associated with improved survival of breast cancer. Breast Cancer Res Treat, 2017. 164(3): p. 689-696.
9. Yeong, J., et al., Prognostic value of CD8+PD-1+ immune infiltrates and PDCD1 gene expression in triple negative breast cancer. J Immunother Cancer, 2019. 7(1): p. 34.
10. Sobral-Leite, M., et al., Cancer-immune interactions in ER-positive breast cancers: PI3K pathway alterations and tumor-infiltrating lymphocytes. Breast Cancer Res, 2019. 21(1): p. 90.
11. Miyoshi, Y., et al., Associations in tumor infiltrating lymphocytes between clinicopathological factors and clinical outcomes in estrogen receptor-positive/human epidermal growth factor receptor type 2 negative breast cancer. Oncol Lett, 2019. 17(2): p. 2177-2186.

The following non-limiting examples are provided for further context of the present disclosure.

### EXAMPLE 1

To evaluate the relationship between radiotherapy benefit and PD-1 expression, we used publicly available gene expression data sets (Servant, N., et al., Search for a gene expression signature of breast cancer local recurrence in young women. Clin Cancer Res, 2012. 18(6): p. 1704-15; Sjostrom, M., et al., Identification and validation of single-sample breast cancer radiosensitivity gene expression predictors. Breast Cancer Res, 2018. 20(1): p. 64; van de Vijver, M. J., et al., A gene-expression signature as a predictor of survival in breast cancer. N Engl J Med, 2002. 347(25): p. 1999-2009). The datasets include patients with early stage breast cancer treated with RT and contain detailed information regarding local recurrence. Analyses were restricted to the first 10 years.

Patients from the SweBCG91RT trial were included and have been described elsewhere (1). In summary, 1178 patients with lymph-node negative (NO) stage I or IIA breast cancer were randomly assigned between 1991 and 1997 to breast conserving surgery with or without whole-breast RT and followed for a median time of 15.2 years. RT was given with tangential opposed fields of 4 or 6 MV photons, with an absorbed dose of 48 to 54 Gy in 24 to 27 fractions, to the remaining breast parenchyma. PD-1 (formal gene name: PDCD1) mRNA was converted to cDNA by reverse transcription after extraction of mRNA, GeneChip Human Exon 1.0 ST Arrays (Thermo Fisher Scientific, South San Francisco, CA) was used to obtain gene expression data (Gene Expression Omnibus with accession number GSE119295) as described previously (2). Briefly, a representative tumor area was marked on a hematoxylin and eosin stained slide. RNA was extracted from 1.5 mm tissue punches using the RNeasy FFPE kit (Qiagen, Hilden, Germany). cDNA was amplified using 20 the Ovation FFPE WTA system (NuGEN, San Carlos, CA). Amplified cDNA was fragmented and labeled using the Encore Biotin Module (NuGEN, San Carlos, CA) and hybridized to GeneChip Human Exon 1.0 ST Arrays (Thermo Fisher Scientific, South San Francisco, CA). Gene expression was normalized using Single Channel Array Normalization. Sample processing was performed in a CLIA-certified clinical operations laboratory (GenomeDx Inc, San Diego, CA).

Clinicopathological variables did not differ significantly between excluded and included patients, except for tumor size which was smaller (pₜ₋ₜₑₛₜ=0.001) among excluded patients (median tumor size 11 mm) compared to included patients (median tumor size 12 mm). In total, 7% of patients received endocrine treatment, 1% received chemotherapy and 0.4% received both endocrine treatment and chemotherapy. The trial and follow-up study were conducted in accordance with the declaration of Helsinki. Informed oral consent was obtained from all patients which was determined appropriate and approved by the Regional Ethical Review Board for the original study and for this study.

Time to ipsilateral breast tumor recurrence (IBTR) as the first event within 10 years from date of diagnosis was used as primary endpoint. Secondary endpoints were time to any recurrence and distant metastasis as the first event within 10 years. Other recurrences and death were considered competing risks for IBTR and any death considered a competing event for any recurrence and distant metastasis. The primary aim was to analyse the interaction between RT and PD-1 mRNA expression regarding risk of IBTR.

Hazard ratios (HRs) were calculated with cause-specific cox proportional hazards regression (Survival package (3)) to reflect the biologic effect of RT in the presence of competing risks. Cumulative incidences were calculated using a competing risks approach (Cmprsk package (4)). All multivariable analyses were adjusted for age, histological grade, tumor size, subtype and CD8+ T cells. PD-1 is mainly expressed on differentiated CD8+ T cells so we adjusted our multivariable analyses for this cell type in order to analyze the independent effect of PD-1 expression. To create a robust CD8+ T cell variable, we used the tool xCell (6) to quantify CD8+ T cell infiltration. We then scaled the values for CD8+ T cells, CD8+effector memory T cells and CD8+central memory T cells and added them for each sample to create the CD8+ T cell variable. To aid in interpretation, the CD8+ T cell variable was then dichotomized using the 50th percentile as cut-off creating groups of CD8High and CD8Low.

PD-1 mRNA was analysed as a continuous variable in the public data sets and for all interaction tests. For the remaining analyses, the PD-1 variable was dichotomized to facilitate interpretation. A cut-off at the 25th lowest percentile of PD-1 expression was used for all RT analyses to define PD-1^{Low} and PD-1^{High}. A predetermined cut-off separating the 25% of patients with the least alleged RT benefit from the rest has been used before (2) and was chosen based on the rate of local recurrence in the Early Breast Cancer Trialists' Collaborative Group meta-analysis where approximately 25% of patients with early, nodenegative breast cancer experienced a local recurrence without RT (7).

The proportional hazards assumption was checked using the Schoenfeld residuals. It was violated for RT and the interaction term RT:PD-1. The hazard ratios for these variables should therefore be interpreted as the mean over the follow-up period of years 0-10. R version 3.6.1 was used for statistical analyses

TABLE 4
Univariable cox regression analysis of PD-1 expression and other clinicopathological variables on risk of IBTR,
any recurrence, distant metastasis and breast cancer death We concluded that increased PD-1 mRNA conferred an
improved prognosis among irradiated patients. Low PD-1 mRNA levels were associated with less benefit from RT
(HR 0.66, CI 95% 0.34-1.27, p = 0.212) compared to high levels (HR 0.30, CI 95% 0.18-0.52, p < 0.001).
The same pattern was seen for any recurrence, distant metastasis and breast cancer death.

| | IBTR | | Any Recurrence | | Distant Metastasis | | Breast cancer death | |
|---|---|---|---|---|---|---|---|---|
| | HR | | HR | | HR | | HR | |
| Variables | (CI 95%) | P value | (CI 95%) | P value | (CI 95%) | P value | (CI 95%) | P value |
| PD1^{High} vs | 0.67 | 0.046 | 0.76 | 0.092 | 0.84 | 0.48 | 0.87 | 0.46 |
| PD1^{Low} | (0.45-0.99) | | (0.55-1.05) | | (0.51-1.37) | | (0.59-1.27) | |
| PD1^{Low} RT | 0.66 | 0.21 | 0.80 | 0.43 | 1.04 | 0.93 | 1.15 | 0.68 |
| vs no RT | (0.34-1.27) | | (0.47-1.38) | | (0.45-2.40) | | (0.59-2.2) | |
| PD1^{High} RT | 0.30 | <0.001 | 0.43 | <0.001 | 0.60 | 0.06 | 0.80 | 0.27 |
| vs no RT | (0.18-0.52) | | (0.29-0.64) | | (0.35-1.03) | | (0.54-1.19) | |
| PD1:RT | 0.005 | 0.031 | 0.034 | 0.09 | 0.02 | 0.18 | 0.014 | 0.051 |
| interaction | (0.001-0.60) | | (0.001-1.71) | | (0.001-6.12) | | (0.001-1.03) | |
| RT | 0.41 | <0.001 | 0.53 | <0.001 | 0.71 | 0.14 | 0.89 | 0.5 |
| | (0.28-0.62) | | (0.39-0.73) | | (0.45-1.12) | | (0.63-1.25) | |
| Grade I | 1.0 (ref) | | 1.0 (ref) | | 1.0 (ref) | | 1.0 (ref) | |
| Grade II | 1.89 | 0.07 | 2 | 0.024 | 2.85 | 0.08 | 2.82 | 0.009 |
| | (0.95-3.79) | | (1.1-3.64) | | (0.889.27) | | (1.36.12) | |
| Grade III | 2.65 | 0.009 | 3.53 | <0.001 | 8.44 | <0.001 | 5.17 | <0.001 |
| | (1.28-5.51) | | (1.9-6.54) | | (2.61-27.33) | | (2.35-11.38 | |
| Luminal | 1.0 (ref) | | 1.0 (ref) | | 1.0 (ref) | | | |
| A | | | | | | | | |
| Luminal | 1.11 | 0.63 | 1.28 | 0.16 | 2.13 | 0.006 | 1.9 | 0.001 |
| B | (0.72-1.71) | | (0.91-1.82) | | (1.24-3.65) | | (1.3-2.77) | |
| HER2+ | 1.3 | 0.47 | 1.64 | 0.07 | 2.94 | 0.005 | 1.49 | 0.244 |
| | (0.64-2.61) | | (0.96-2.80) | | (1.38-6.26) | | (0.76-2.92) | |
| Triple | 1.59 | 0.14 | 1.97 | 0.005 | 4.45 | <0.001 | 2.56 | 0.001 |
| negative | (0.85-2.96) | | (1.22-3.19) | | (2.36-8.41) | | (1.49-4.41) | |
| Size (mm) | 1.00 | 0.87 | 1.04 | 0.003 | 1.07 | <0.001 | 1.04 | 0.012 |
| | (0.97-1.04) | | (1.01-1.07) | | (1.04-1.11) | | (1.01-1.07) | |
| Age (years) | 0.97 | 0.005 | 0.97 | <0.001 | 0.97 | 0.003 | 1.01 | 0.40 |
| | (0.95-0.99) | | (0.96-0.99) | | (0.94-0.99) | | (0.99-1.03) | |
| Systemic | 0.40 | 0.068 | 0.74 | 0.324 | 0.92 | 0.85 | 0.68 | 0.299 |
| treatment | (0.15-1.07) | | (0.40-1.35) | | (0.40-2.12) | | (0.34-1.4) | |
| CD8^{High} vs | 0.74 | 0.116 | 0.79 | 0.125 | 0.67 | 0.077 | 0.72 | 0.061 |
| CD8^{Low} | (0.51-1.08) | | (0.59-1.07) | | (0.42-1.04) | | (0.52-1.01) | |

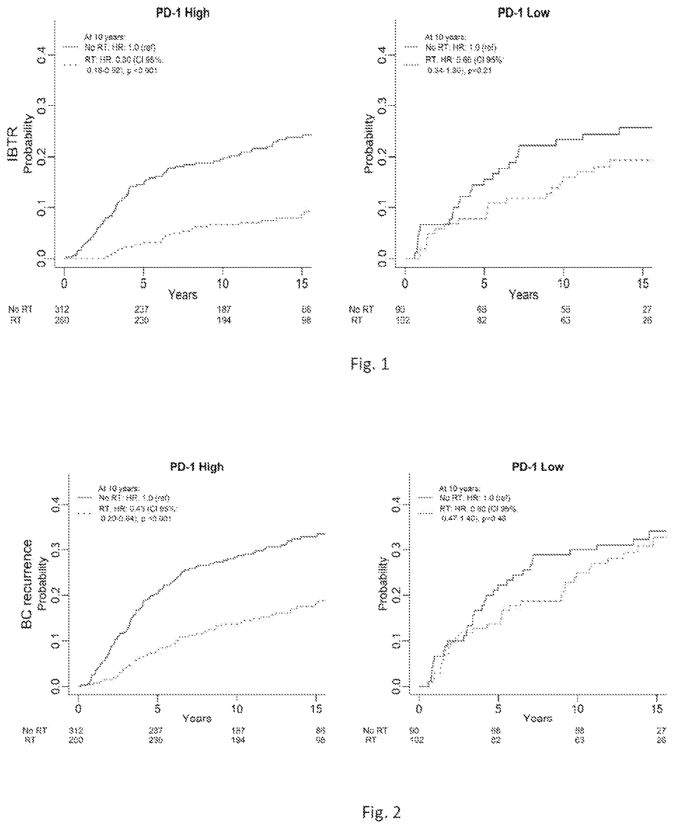

With IBTR as the dependent variable, the interaction test between RT and PD-1 expression was significant in unadjusted (p=0.031) and multivariable (p=0.016) analysis adjusted for subtype, age and histological grade (Table 4). The multivariable interaction test for any recurrence (p=0.045) and distant metastasis (p=0.12) also indicated an increased RT benefit with increasing PD-1 mRNA. FIGS. 1 and 2 show the cumulative incidence of IBTR and BC recurrence for patients that express high and low levels of PD-1, with or without whole breast radiation treatment following breast conserving surgery.

### References to Example 1

1. Malmstrom, P., et al., Breast conservation surgery, with and without radiotherapy, in women with lymph node-negative breast cancer: a randomised clinical trial in a population with access to public mammography screening. Eur J Cancer, 2003. 39(12): p. 1690-7.
2. Sjostrom, M., et al., Clinicogenomic Radiotherapy Classifier Predicting the Need for Intensified Locoregional Treatment After Breast-Conserving Surgery for Early-Stage Breast Cancer. J Clin Oncol, 2019. 37(35): p. 3340-3349.
3. Therneau, T. M., A Package for Survival Analysis in S. 2015.
4. Gray, B., cmprsk: Subdistribution Analysis of Competing Risks. 2019.
6. Aran, D., Z. Hu, and A. J. Butte, xCell: digitally portraying the tissue cellular heterogeneity landscape. Genome Biol, 2017. 18(1): p. 220.
7. Clarke, M., et al., Effects of radiotherapy and of differences in the extent of surgery for early breast cancer on local recurrence and 15-year survival: an overview of the randomised trials. Lancet, 2005. 366(9503): p. 2087-106.

All analyses are performed for local recurrences with 10 years of follow-up unless otherwise stated.

### EXAMPLE 2

### Generation of Threshold

PD-1 expression level can be analyzed in relation to a reference group. The reference group is a group of patients that is similar to the patient being treated (for example women with early breast cancer). In the reference group, the PD-1 expression level of the members of the group has been quantified and ranked. Various percentiles between 5th and 90^{th} percentiles were chosen as a threshold to determine whether it is beneficial to provide intensified treatment to a patient. By doing this, it was possible to determine a) that there is a benefit from the process over simply guessing whether a subject would benefit from an intensified therapy and b) what range or percent of the reference group can be used to define a low level of PD-1 (e.g., can it be lower or greater than 25%, and if so, how much and how does that influence other aspects of the method). The following experiments were performed to determine a likelihood that a subject will benefit from radiation therapy. Based on various thresholds, radiotherapy benefit for patients above and below the threshold was compared. The results are presented in the following tables (5-*):

mRNA Thresholds

TABLE 5

| | Radiotherapy | | Radiotherapy | | | |
|---|---|---|---|---|---|---|
| | benefit for | | benefit for | | | |
| | patients above | | patients below | | | |
| Percentile | percentile | | percentile | | interaction | test |
| cut-off | HR | pval | HR | pval | HR | pval |
| 5 | 0.397 | <0.0001 | 0.799 | 0.7455 | 2.163 | 0.2861 |
| 10 | 0.365 | <0.0001 | 0.980 | 0.9693 | 2.712 | 0.0819 |
| 15 | 0.333 | <0.0001 | 1.079 | 0.8724 | 3.253 | 0.0252 |
| 20 | 0.334 | <0.0001 | 0.633 | 0.2035 | 1.907 | 0.1447 |
| 25 | 0.301 | <0.0001 | 0.667 | 0.2228 | 2.223 | 0.0638 |
| 30 | 0.242 | <0.0001 | 0.770 | 0.3832 | 3.194 | 0.0069 |
| 40 | 0.243 | <0.0001 | 0.639 | 0.0975 | 2.625 | 0.0254 |
| 50 | 0.299 | 0.0008 | 0.498 | 0.0060 | 1.660 | 0.2509 |
| 60 | 0.282 | 0.0008 | 0.500 | 0.0055 | 1.772 | 0.2057 |
| 70 | 0.288 | 0.0036 | 0.467 | 0.0013 | 1.610 | 0.3299 |
| 80 | 0.280 | 0.0227 | 0.441 | 0.0002 | 1.561 | 0.4593 |
| 90 | 0.320 | 0.0926 | 0.424 | 0.0001 | 1.254 | 0.7500 |

Significant p values are bolded

As shown in table 5, PD-1 mRNA thresholds at the 50th percentile or above do not work to determine a likelihood that a subject will benefit from radiation therapy. No significant interaction is detected and both the group above and below the threshold show a significant benefit from postoperative radiotherapy. On the other hand, it works when PD-1 mRNA thresholds were set at 5^{th}, 10^{th}, 15^{th}, 20^{th}, 25^{th}, 30^{th} percentiles. The data shows that strongest interaction (p=0.0069) is observed when the threshold is set to 30^{th} percentile.

At or below the 15^{th} percentile (HR: 1.08) vs at or below the 30^{th} percentile (HR: 0.77) vs at or below the 50^{th} percentile (HR: 0.50) vs above the 50^{th} percentile (HR: 0.30).

i. Age <50, Age>70

Interaction between radiotherapy and continuous PD-1 mRNA variable was checked based on age groups. As shown in table 6, the interaction is mainly driven by patients of age 50-60 years.

TABLE 6

| Age group | HR | Pval |
|---|---|---|
| 30-50 | 0.633 | 0.2648 |
| 50-60 | 0.366 | 0.0247 |
| 55-65 | 0.489 | 0.0501 |
| 60-70 | 0.667 | 0.2788 |
| 70-80 | 0.486 | 0.5806 |

ii. Tumor Subtype

The interaction between PD-1 mRNA expression and tumor subtype was checked. As shown in table 7, the interaction is mainly driven by Luminal B tumors.

TABLE 7

| Subtype | HR | Pval |
|---|---|---|
| HER2+ | 0.392 | 0.1858 |
| Luminal A | 0.9 | 0.7149 |
| Luminal B (HER2-) | 0.207 | <0.001 |
| Triple Negative | 0.85 | 0.8293 |

PD1 mRNA is predictive for locoregional recurrences with 15 years of follow-up (p value=0.01).

### EXAMPLE 3

This example assess how local recurrence is affected by PD1-mRNA expression in RT treated early breast cancer.

### PD-1 Was the Checkpoint Molecule Most Strongly Associated With a Decreased Risk of Local Recurrence

Three publicly available gene expression cohorts of early-stage breast cancer treated with adjuvant RT were analyzed: Servant (n=343)[1], Sjostrom (n=172)[2] and van de Vijver (n=295)[3]. Analyses were restricted to the first 10 years and adjusted for all the following when available: age, ESR1, MKI67, CD8+ T cells, and lymph node status. Age and lymph node status were not available for the Sjostrom cohort. For the van de Vijver cohort, overall survival information was available, and this endpoint was also included in the analysis. As seen in Table 8, in the three public RT treated cohorts representing a total of 809 patients, PD-1 was the checkpoint molecule most strongly associated with a decreased risk of local recurrence (Servant=HR 0.74, CI 95% 0.55-0.98, p=0. 035; van de Vijver=HR 0.68, CI 95% 0.39-1.19, p=0.18; Sjostrom=HR 0.65, CI 95% 0.44-0.95, p=0.025, Table 8). In the van de Vijver cohort (n=294), PD-1 mRNA was also prognostic for overall survival (HR 0.55, CI 95% 0.40-0.75, p<0.001).

TABLE 8
Prognostic effect of checkpoint molecules expression in public RT-treated van de
Vijver, Servant and Sjöström data sets.
The variables are standardized. The hazard ratios therefore represent the change with an
increase with one standard deviation. Additional covariates included in the models:
The Servant data set: age, CD8+ T cells, ESR1, MKI67, lymph node status.
The Sjöström data set: CD8+ T cells, ESR1, MKI67. The Van de Vijver data set:
age, lymph node status, grade, CD8+ T cells and ESR1. CD8+ T cell content was
estimated uapproaches, and has shown accuracy in quantifying T cells[5].
sing xCell[4], which combines gene set enrichment and deconvolution

| | van de Vijver | | van de Vijver | | Servant | | Sjöström | |
|---|---|---|---|---|---|---|---|---|
| | (local recurrence) | | (overall survival) | | (local recurrence) | | (local recurrence) | |
| Checkpoint | HR | | HR | | HR | | HR | P |
| molecule | (95% CI) | P value | (95% CI) | value | (95% CI) | value | (95% CI) | value |
| PDCD1 | 0.68 | 0.176 | 0.55 | 0.001 | 0.74 | .035 | 0.65 | 0.025 |
| (PD-1) | (0.39-1.19) | | (0.40-0.75) | | (0.55-0.98) | | (0.44-0.95) | |

The Matched Metabric Cohort Shows that PD-1 mRNA Expression Was Associated With an Increased RT Benefit Regarding Overall Survival

The RT predictive effect of PD-1 mRNA expression on overall survival was further analyzed in the Metabric cohort[6, 7] (median follow-up time among deceased and living patients: 7.2 years and 13.2, respectively). Starting with the cohort of N=785 patients operated with breast-conserving surgery, patients treated and not treated with RT were matched 1:1 to clinical variables (Table 9), using OptMatch[8] and RItools[9,10] The resulting matched cohort consisted of N=144 patients. Analysis in this matched cohort was adjusted for subtype, age, lymph node status, systemic therapy, Nottingham prognostic index and CD8+ T cells. In the resulting 1:1 matched Metabric cohort of 144 patients, PD-1 mRNA expression was associated with an increased RT benefit regarding overall survival (p_{interaction}=0.038)

TABLE 9
Multivariable analysis of effect of RT on overall mortality depending on PD-1
expression and distribution of variables depending on RT treatment in the matched Metabric cohort.
Increased PD-1 mRNA expression was predictive of increased RT benefit regarding overall mortality. Patients
treated with breast-conserving surgery and RT in the Metabric cohort were matched 1:1 with patients treated
with breast-conserving surgery only. The interaction between RT and PD-1 expression was analyzed using the
continuous PD-1 variable. The cutoff used to define PD-1High/PD-1Low was the 25th percentile of PD-1
expression.

| | | | Distribution of variables | | |
|---|---|---|---|---|---|
| | Multivariable cox regression analysis | | | | P |
| Variables | HR (95% CI) | P value | No RT (n=72) | RT (n = 72) | value *** |
| PD-1^{Low} | 1.0 (ref) | | 17 (23.6%) | 19 (26.4%) | 0.85 |
| PD-1^{High} | 0.59 (0.32-1.10) | 0.096 | 55 (76.4%) | 53 (73.6%) | |
| PD-1 continuous* | 0.69 0.25-1.09) | 0.48 | 5.63 (0.36)** | 5.58 (0.37)** | 0.45 |
| RT | 0.63 (0.38-1.04) | 0.068 | 0 (100%) | 72 (100%) | |
| PD-1:RT interaction | | 0.038 | | | |
| Nottingham | 1.14 (0.81-1.60) | 0.47 | 3.5 (1.24)** | 3.47 (1.21)** | 0.88 |
| prognostic index | | | | | |
| Lymph nodes positive | 0.99 (0.87-1.13) | 0.92 | 0 (2.71)** | 0 (2.66)** | 0.8 |
| Age (years) | 1.07 (1.04-1.10) | <0.001 | 62.82 (13.31)** | 62.71 (12.56)** | 0.96 |
| Luminal A | 1.0 (ref) | | 31 (43.1%) | 31 (43.1%) | |
| Luminal B | 1.30 (0.63-2.67) | 0.48 | 19 (26.4%) | 19 (26.4%) | |
| HER2+ | 1.07 (0.35-3.23) | 0.91 | 5 (6.9%) | 5 (6.9%) | |
| Basal | 1.71 (0.51-5.78) | 0.38 | 6 (8.3%) | 6 (8.3%) | |
| Claudin-low | 0.74 (0.23-2.38) | 0.62 | 6 (8.3%) | 6 (8.3%) | |
| Normal | 0.92 (0.23-2.38) | 0.90 | 5 (6.9%) | 5 (6.9%) | |
| CD8Low | 1.0 (ref) | 0.028 | 32 (44.4%) | 40 (55.6%) | 0.24 |
| CD8High | 2.08 (1.08-3.99) | | 40 (55.6%) | 32 (44.4%) | |
| No chemotherapy | 1.0 (ref) | 0.005 | 65 (90.3%) | 65 (90.3%) | 1 |
| Chemotherapy | 4.44 (1.58-12.47) | | 7 (9.7%) | 7 (9.7%) | |
| No hormone therapy | 1.0 (ref) | 0.94 | 30 (41.7%) | 31 (43.1%) | 1 |
| Hormone Therapy | 1.02 (0.57-1.82) | | 42 (58.3%) | 41 (56.9%) | |

*The continuous and dichotomized PD-1 variables were not included in the same model (i.e., only one PD-1 variable was tested at a time).
**Median (SD).
***Chi-square, t-test or linear test for trend.

1. Servant, N., et al., Search for a gene expression signature of breast cancer local recurrence in young women. Clin Cancer Res, 2012. 18(6): p. 1704-15.
2. Sjostrom, M., et al., Identification and validation of single-sample breast cancer radiosensitivity gene expression predictors. Breast Cancer Res, 2018. 20(1): p. 64.
3. van de Vijver, M. J., et al., A gene-expression signature as a predictor of survival in breast cancer. N Engl J Med, 2002. 347(25): p. 1999-2009.
4. Aran, D., Z. Hu, and A. J. Butte, xCell: digitally portraying the tissue cellular heterogeneity landscape. Genome Biol, 2017. 18(1): p. 220.
5. Sturm, G., et al., Comprehensive evaluation of transcriptome-based cell-type quantification methods for immuno-oncology. Bioinformatics, 2019. 35(14): p. i436-i445.
6. https://www.cbioportal.org/study/summary?id=brcametabric.
7. Curtis, C., et al., The genomic and transcriptomic architecture of 2,000 breast tumours reveals novel subgroups. Nature, 2012. 486(7403): p. 346-52.
8. Hansen B B, K. S., Optimal full matching and related designs via network flows. Journal of Computational and Graphical Statistics, 15(3), 609-627, 2006.
9. Bowers, B. B. H. a. J., Covariate balance in simple, stratified and clustered comparative studies. Statistical Science. 23(2):219-236., 2008.
10. Jake Bowers, M. F., and Ben Hansen, RItools:Randomization Inference Tools. R package version 0.1-17. 2019

In some embodiments, any 1, 2, 3, 4, 5, 6, 7, 8, 9, or more of the variables tested and shown to be effective in the above examples can be combined with the PD-1 based methods provided herein, either on their own (in combination with PD-1) or with other variables withing the disclosure. For example, HER2, Luminal, CD8, etc. can either in combination or separately be used to determine if radiotherapy will be effective—so one can determine if the subject should receive an aggressive (or intense) therapy, or no therapy at all.

While the invention has been described with reference to specific exemplary embodiments, the description is in general only intended to illustrate the inventive concept and should not be taken as limiting the scope of the invention. The invention is generally defined by the claims.

1. A method for treating breast cancer (both invasive and in situ) comprising the steps:
a) obtaining a tissue sample of a tumour from a breast cancer patient,
b) determining the expression levels of PD-1 mRNA in the sample,
c) determining that the expression level is below a threshold level,
d) providing intensified treatment as intensified radiotherapy treatment, intensified systemic therapy or mastectomy to the patient.

2. The method of claim 1 where the intensified treatment comprises intensified radiotherapy treatment.

3. (canceled)

4. (canceled)

5. The method of claim 1 where the patient has been subjected to breast conserving surgery or total mastectomy.

6. The method of claim 1 where the breast cancer is an early stage invasive breast cancer or breast cancer in situ.

7. The method of claim 1 where the expression level of PD-1 is determined by detecting the amount of PD-1 mRNA in the sample.

8. (canceled)

9. (canceled)

10. A PD-1 mRNA-binding nucleotide for use in the diagnosis of breast cancer, where the nucleotide is used for quantifying the level of PD-1 that is expressed in a breast cancer sample, and where low expression of PD-1 indicates that the patients belong to a patient subgroup where intensified radiotherapy treatment is needed.

11. (canceled)

12. A method of treating a subject, the method comprising:
identifying an incremental risk to a subject with invasive breast cancer or in situ breast cancer of a local or regional recurrence of an invasive breast cancer based on a level of PD-1 in a sample of an invasive breast cancer in the subject; and
administering an intensified breast cancer therapy to the subject based upon the incremental risk, wherein a higher incremental risk will increase:
a) a likelihood of an aggressive breast cancer therapy that is at least more than what would be recommended by the NCCN;
b) the aggressiveness of the aggressive breast cancer; or
c) both a) and b).

13. (canceled)

14. A method of treating a subject, the method comprising:
identifying a subject with invasive breast cancer that has a low level of PD-1; and
administering an intensified treatment to the invasive breast cancer.

15. (canceled)

16. A method for preventing an invasive breast cancer recurrence in a subject, the method comprising:
providing a cancer tissue sample from a subject who has invasive breast cancer;
analyzing the cancer tissue sample for a level of PD-1; and
administering an intensified treatment if the cancer tissue sample has a low level of PD-1.

17. A method for preventing an invasive breast cancer recurrence in a subject, the method comprising:
receiving an intensified treatment if a cancer has a low level of PD-1.

18. (canceled)

19. (canceled)

20. The method of claim 16, wherein low level of PD-1 is defined by a comparison of PD-1 levels from the tissue sample to a control sample that does not include invasive cancer or to an internal control in the sample.

21.-25. (canceled)

26. The method of claim 16, wherein the intensified treatment includes at least one of: intensified radiotherapy treatment, systemic therapy or mastectomy.

27. The method of claim 26, wherein treating the subject with intensified radiotherapy denotes a therapy above the guidelines in the NCCN, ESMO, Clinical Practice Recommendations Australia, or NICE guideline, ignoring the PD-1 marker state.

28. (canceled)

29. The method of claim 26, wherein treating the subject with intensified radiotherapy denotes a dose of at least one of: 67 Gy or more, add a boosting dose to a standard recommended treatment for the subject when the standard recommended treatment does not include a boosting dose, increase a boosting dose beyond the standard amount for the subject, increase the fraction dose on a per fraction basis above the standard for the subject, increase the number of fractions of a recommended dose above the standard for the subject.

30. The method of claim 16, wherein low levels of PD-1 denotes the subject has PD-1 levels in a lowest quartile of PD-1 levels of a population of subjects having invasive breast cancer, relative to a set of one or more selected expression levels of housekeeping genes.

31. (canceled)

32. (canceled)

33. The method of claim 26, wherein, when radiation is administered, it is administered at:
a) (for a subject with no boost otherwise recommended) a biologically effective dose of (BED) of 73 Gy or more with a tumor alpha/beta ratio of 5, or a BED of 78 Gy or more with a tumor alpha/beta ratio of 4, or a BED of 87 Gy or more with a tumor alpha/beta ratio of 3, or a BED of 104 Gy or more with a tumor alpha/beta ratio of 2; or
b) (for a subject with a boost otherwise recommended) a biologically effective dose of (BED) of 93 Gy or more with a tumor alpha/beta ratio of 5 or a BED of 100 Gy or more with a tumor alpha/beta ratio of 4 or a BED of 111 Gy or more with a tumor alpha/beta ratio of 3 or a BED of 133 Gy or more with a tumor alpha/beta ratio of 2 for patients who are recommended a boost according to the current guidelines,
wherein BED is a measure of the true biological dose delivered by a combination of dose per fraction (d) and number of fractions (n) to a tissue characterized by a specific radiosensitivity (alfa/beta ratio): BED=nd[1+d/(alfa/beta)].

34. The method of claim 16, wherein a level of PD-1 is analyzed as a continuous metric so that a continuous risk assessment is further provided to the subject.

35. The method of claim 16, wherein the intensified treatment denotes at least one of: intensified radiotherapy treatment, systemic therapy, mastectomy, the additional use of a sensitizer to another therapy; a therapy above a level set by a guideline selected from a NCCN, ESMO, Clinical Practice Recommendations Australia, or NICE guideline for the subject's remaining indicators, or any combination thereof.

36. The method of claim 16, wherein the level of PD-1 is determined by at least one of: laser capture, microdissection, RT-PCR, QPCR, PCR, deep sequencing, RNA-seq, a microarray assay, normalized and non-normalized probes, and NanoString.

37. (canceled)

38. The method of claim 16, wherein recurrence is a local and/or regional recurrence.

39. (canceled)

40. The method of claim 16, wherein the sample comprises a core biopsy sample, a fine-needle aspiration (FNA) sample, an excisional biopsy sample or a sample from surgery.

41. (canceled)

42. The method of claim 1, wherein the intensified treatment comprises a therapy above the guidelines in the NCCN, ESMO, Clinical Practice Recommendations Australia, or NICE guideline, ignoring the PD-1 marker state.

43. The method of claim 1, wherein the intensified radiotherapy treatment comprises at least one of: a dose of 67 Gy or more, add a boosting dose to a standard recommended treatment for the subject when the standard recommended treatment does not include a boosting dose, increase a boosting dose beyond the standard amount for the subject, increase the fraction dose on a per fraction basis above the standard for the subject, increase the number of fractions of a recommended dose above the standard for the subject.

44. The method of claim 1, wherein
(a) for a subject with no boost otherwise recommended, the intensified radiotherapy treatment is whole breast external radiotherapy, partial breast radiotherapy or brachytherapy or a combination thereof, with a biologically effective dose of (BED) of 73 Gy or more with a tumor alpha/beta ratio of 5 or a BED of 78 Gy or more with a tumor alpha/beta ratio of 4 or a BED of 87 Gy or more with a tumor alpha/beta ratio of 3 or a BED of 104 Gy or more with a tumor alpha/beta ratio of 2; or
(b) for a subject with a boost otherwise recommended, the intensified radiotherapy treatment is one or more of whole breast external radiotherapy, partial breast radiotherapy or brachytherapy or a combination thereof, with a biologically effective dose of (BED) of 93 Gy or more with a tumor alpha/beta ratio of 5 or a BED of 100 Gy or more with a tumor alpha/beta ratio of 4 or a BED of 111 Gy or more with a tumor alpha/beta ratio of 3 or a BED of 133 Gy or more with a tumor alpha/beta ratio of 2 for patients who are recommended a boost according to the current guidelines.


SEQUENCE LISTING (4 sequences)

SEQ ID NO: 1 - DNA, 2097 bp, Unknown
  cDNA
gctcacctccgcctgagcagtggagaaggcggcactctggtggggctgctccaggcatgcagatcccacaggcgccctggccagtcgtctgggcggtgctacaactgggctggcggccaggatggttcttagactccccagacaggccctggaacccccccaccttctccccagccctgctcgtggtgaccgaaggggacaacgccaccttcacctgcagcttctccaacacatcggagagcttcgtgctaaactggtaccgcatgagccccagcaaccagacggacaagctggccgccttccccgaggaccgcagccagcccggccaggactgccgcttccgtgtcacacaactgcccaacgggcgtgacttccacatgagcgtggtcagggcccggcgcaatgacagcggcacctacctctgtggggccatctccctggcccccaaggcgcagatcaaagagagcctgcgggcagagctcagggtgacagagagaagggcagaagtgcccacagcccaccccagcccctcacccaggccagccggccagttccaaaccctggtggttggtgtcgtgggcggcctgctgggcagcctggtgctgctagtctgggtcctggccgtcatctgctcccgggccgcacgagggacaataggagccaggcgcaccggccagcccctgaaggaggacccctcagccgtgcctgtgttctctgtggactatggggagctggatttccagtggcgagagaagaccccggagccccccgtgccctgtgtccctgagcagacggagtatgccaccattgtctttcctagcggaatgggcacctcatcccccgcccgcaggggctcagctgacggccctcggagtgcccagccactgaggcctgaggatggacactgctcttggcccctctgaccggcttccttggccaccagtgttctgcagaccctccaccatgagcccgggtcagcgcatttcctcaggagaagcaggcagggtgcaggccattgcaggccgtccaggggctgagctgcctgggggcgaccggggctccagcctgcacctgcaccaggcacagccccaccacaggactcatgtctcaatgcccacagtgagcccaggcagcaggtgtcaccgtcccctacagggagggccagatgcagtcactgcttcaggtcctgccagcacagagctgcctgcgtccagctccctgaatctctgctgctgctgctgctgctgctgctgctgcctgcggcccggggctgaaggcgccgtggccctgcctgacgccccggagcctcctgcctgaacttgggggctggttggagatggccttggagcagccaaggtgcccctggcagtggcatcccgaaacgccctggacgcagggcccaagactgggcacaggagtgggaggtacatggggctggggactccccaggagttatctgctccctgcaggcctagagaagtttcagggaaggtcagaagagctcctggctgtggtgggcagggcaggaaacccctccacctttacacatgcccaggcagcacctcaggccctttgtggggcagggaagctgaggcagtaagcgggcaggcagagctggaggcctttcaggcccagccagcactctggcctcctgccgccgcattccaccccagcccctcacaccactcgggagagggacatcctacggtcccaaggtcaggagggcagggctggggttgactcaggcccctcccagctgtggccacctgggtgttgggagggcagaagtgcaggcacctagggccccccatgtgcccaccctgggagctctccttggaacccattcctgaaattatttaaaggggttggccgggctcccaccagggcctgggtgggaaggtacaggcgttcccccggggcctagtacccccgccgtggcctatccactcctcacatccacacactgcacccccactcctggggcagggccaccagcatccaggcggccagcaggcacctgagtggctgggacaagggatcccccttccctgtggttctattatattataattataattaaatatgagagcatgctaa

SEQ ID NO: 2 - DNA, 20 bp, Unknown
  Synthetic Primer
cgtggcctatccactcctca

SEQ ID NO: 3 - DNA, 20 bp, Unknown
  Synthetic Primer
atcccttgtcccagccactc

SEQ ID NO: 4 - protein, 287 aa, Unknown
  Mammal
MQIPQAPWPVVWAVLQLGWRPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVVGVVGGLLGSLVLLVWVLAVICSRAARGTIGARRTGQPLEDPSAVPVFSVDYGELDFQWREKTPEPPVPCVPEQTEYATIVFPSGMGTSSPARRGSADGPRSAQPLRPEDGHCSWPL